(19) United States
(12) Patent Application Publication
(10) Pub. No.: US 2026/0199520 A1
(43) Pub. Date: Jul. 16, 2026

(54) Lipid Nanoparticle Modified with a Targeting Moiety for Targeted Delivery

(71) Applicant: NanoVation Therapeutics Inc., Vancouver (CA)

(72) Inventors: Rupsa Gupta, Vancouver (CA); Lokesh Narsineni, Vancouver (CA); Daniel Kurek, Vancouver (CA); Dominik Witzigmann, Binzen (DE); Jayesh Kulkami, Vancouver (CA)

(73) Assignee: NanoVation Therapeutics Inc., Vancouver, BC (CA)

(21) Appl. No.: 19/136,389

(22) PCT Filed: Dec. 8, 2023
(86) PCT No.: PCT/CA2023/051632

§ 371 Date: Jun. 6, 2025

Related U.S. Application Data
(60) Provisional application No. 63/431,177, filed on Dec. 8, 2022.
(60) Provisional application No. 63/588,171, filed on Oct. 5, 2023.

(51) Int. Cl. A61K 48/00; A61K 9/127; A61K 47/24; A61K 47/26; A61K 47/69

(52) U.S. Cl. CPC A61K 48/0033; A61K 9/127; A61K 47/24; A61K 47/26; A61K 47/6911

(57) ABSTRACT

Provided herein is a lipid nanoparticle encapsulating nucleic acid and having at least 30 mol % neutral lipid, a sterol or derivative thereof and a targeting moiety anchored in a lipid layer thereof via a lipophilic moiety. Further provided are methods of using the lipid nanoparticles for targeted delivery in vivo. Such lipid nanoparticle may exhibit significantly improved delivery and targeting to extrahepatic tissues and/or organs.

### TECHNICAL FIELD

The present disclosure relates to lipid nanoparticle formulations for the delivery of nucleic acid.

### BACKGROUND

Lipid nanoparticle (LNP) formulations represent a significant advancement in the field of nucleic acid delivery. An early example of a lipid nanoparticle product approved for clinical use is Onpattro™ developed by Alnylam. Onpattro™ is a lipid nanoparticle-based short interfering RNA (siRNA) drug for the treatment of polyneuropathies induced by hereditary transthyretin amyloidosis. The success of this LNP delivery system paved the way for the clinical development of the leading LNP-based COVID-19 mRNA vaccines.

The Onpattro™ LNP formulation consists of four main lipid components, namely: ionizable amino lipid (DLin-MC3-DMA or “MC3” (dilinoleyl-methyl-4-dimethylaminobutyrate)), distearoylphosphatidylcholine (DSPC), cholesterol, and polyethylene glycol conjugated lipids (PEG-lipids) at respective molar amounts of 50/10/38.5/1.5. Onpattro™ is still considered the gold standard for comparison in studies of LNP-mediated efficacy and current approaches to the design of LNPs for use in the clinic make few deviations from the four-component system.

Of these four components, the ionizable lipid makes up the bulk of the Onpattro™ formulation and is present at 50 mol %. The ionizable lipid is considered important for the in vitro and in vivo activity of the LNP system and therefore most work in the field has focused primarily on improving the potency of this lipid component. The ionizable lipid is typically an amino lipid and has been carefully designed so that it is charged at low pH and near neutral at physiological pH. This allows for electrostatic interactions between the lipid and the negatively charged nucleic acid during initial formulation. Since the ionizable lipid is near neutral at physiological pH, toxicity and renal clearance is reduced. After cellular uptake by endocytosis, the acidic environment of the endosome leads to an increase in the net positive charge of the ionizable amino lipids, which promotes fusion with the anionic lipids of the endosomal membrane and subsequent membrane destabilization and release of the nucleic acid-based therapeutics into the cytoplasm to exert their effects.

With respect to the remaining three lipid components, the PEG-lipid is well known for improving circulation longevity of the LNP and cholesterol functions to stabilize the particle. Generally, however, comparatively less attention has been devoted to studying neutral lipid components in LNPs beyond their structural role.

The liver is the primary organ in which the Onpattro™ four-component LNP accumulates after intravenous administration. While delivery to the liver has therapeutic potential, the ability of LNPs to accumulate in organs and tissues beyond the liver would greatly expand the clinical utility of these delivery systems. Extrahepatic delivery could improve treatment and/or prevention of cancer, cardiovascular disease, infectious disease, among other diseases.

However, efforts to target tissues beyond the liver (extrahepatic organs and tissues) using the intravenous route have been met with less success. In order to improve LNP delivery to extrahepatic tissues, the particles should exhibit enhanced circulation lifetimes. As noted, traditional approaches to achieve this involve designing LNPs with a long-lasting PEG coating, often referred to as “stealth” liposomes. Nonetheless, the inclusion of PEG-lipids in LNPs often results in transfection potencies that are low. Alternatively, the incorporation of various permanently positively charged lipids can enhance transfection in a number of extrahepatic tissues following i.v. administration. However, such lipids can be toxic, potentially limiting clinical applications.

Studies have found that DSPC and cholesterol contribute to the stable encapsulation of siRNA in LNPs (Kulkarni et al., 2019, Nanoscale, 11:21733-21739). Despite these findings, subsequent in vivo studies by another group failed to show any clear benefit resulting from adjusting the levels of DSPC in LNPs to improve the extra-hepatic delivery of siRNA. These studies examined extrahepatic siRNA gene silencing in vivo with Onpattro™-type LNPs (MC3/Chol/DSPC/PEG-DMP) incorporating DSPC at 10 and 40 mol % (Ordobadi, 2019, “Lipid Nanoparticles for Delivery of Bioactive Molecules”, A Thesis Submitted in Partial Fulfillment of the Requirements for theDegree of Doctor of Philosophy, The University of British Columbia). It was shown that the 10 mol % DSPC Onpattro™ formulation had similar liver accumulation and blood circulation lifetimes as 40 mol % DSPC formulations (MC3/Chol/DSPC/PEG-DMG; 18.5/40/40/1.5 mol %). Further, the 40 mol % DSPC siRNA-containing LNP (siRNA-LNP) only performed comparably to 10 mol % DSPC formulations in bone marrow gene silencing.

The conjugation of various targeting moieties to an siRNA itself or their inclusion as part of a formulation as a surface modification has been examined by various groups as an alternative to passive targeting. However, since the Onpattro™ four-component LNP formulation largely accumulates in liver (hepatic) tissues, most ligand targeting strategies have focused on improving delivery to hepatocytes. For example, Chen et al., (2014, Journal of Controlled Release, 196:106-112) describes the use of LNPs with a hepatocyte-specific targeting ligand, GalNAc-PEG, to improve hepatic gene silencing by increasing uptake of the functionalized LNPs in the liver. WO 2010/144740 describes 4-component liposomal formulations having ionizable lipid (MC3)/DSPC/chol/PEG-lipid in which FVII siRNA silencing in the liver was improved by the addition of GalNAc to the surface of liposomes. Mannose-containing LNPs also have been used to target HepG2 liver cells.

Despite these previous efforts, there remains a need in the art to improve the targeted delivery of nucleic acid to the liver and/or extrahepatic tissues or organs using LNPs.

### SUMMARY

It has been found by the inventors that surprising improvements in nucleic acid expression in certain tissues and/or organs can be achieved by using a lipid nanoparticle (LNP) with elevated levels of neutral lipid in combination with a moiety on the LNP surface for binding a sub-set of target cells. The inventive LNPs described herein can thus employ two levels of targeting, namely targeting to a desired tissue or organ by the inclusion of elevated levels of neutral lipids and active targeting to cell types of interest having a surface receptor that binds to the targeting moiety on the LNP.

In certain advantageous examples herein, the disclosure provides a lipid nanoparticle (LNP) comprising three or four lipid components for the delivery of nucleic acid. The three or four lipid components include an ionizable lipid, a neutral lipid, such as a phospholipid, a sterol and optionally a hydrophilic polymer-lipid conjugate. In particular, the neutral lipid is present at a content that is higher than that of conventional LNPs, such as at least 20 mol %, at least 30 mol %, at least 36 mol % or at least 40 mol % (relative to total lipid content of the LNP).

According to one aspect of the disclosure, there is provided a lipid nanoparticle comprising: (i) a nucleic acid; (ii) a neutral lipid content of greater than 35 mol %; (iii) an ionizable lipid content of from 5 mol % to 50 mol %; (iv) a sterol or a derivative thereof; and (v) a targeting moiety linked to a lipophilic moiety that is present in a lipid layer of the nanoparticle, the targeting moiety optionally linked to the lipophilic moiety via a linker, wherein each mol % is relative to a total lipid content of the lipid nanoparticle, and wherein the lipid nanoparticle comprises a core, the core optionally comprising an electron dense region and an aqueous portion, and wherein the core is surrounded at least partially by the lipid layer, as visualized by cryogenic electron microscopy (cryo-EM).

According to another aspect of the disclosure, there is provided a lipid nanoparticle encapsulating nucleic acid and having at least 38 mol % neutral lipid, a sterol or derivative thereof, and a targeting moiety anchored in a lipid bilayer or monolayer thereof via a lipophilic moiety, wherein the targeting moiety is present at less than 2.5 mol % and wherein a linker is optionally present between the lipophilic moiety and the targeting moiety.

According to one embodiment, the linker is a hydrophilic polymer that is conjugated at its one end to the lipophilic moiety and at its other end to the targeting moiety.

According to one embodiment, the lipid nanoparticle is produced by ethanol injection comprising a step of lowering the pH of a solution external to the nanoparticle after its formation, thereby producing the core comprising the electron dense region and the aqueous portion.

In another embodiment, the phosphatidylcholine lipid is distearoylphosphatidylcholine (DSPC), dioleoylphosphatidylcholine (DOPC), 1-palmitoyl-2-oleoyl-phosphatidylcholine (POPC), dimyristoyl-phosphatidylcholine (DMPC) or dipalmitoyl-phosphatidylcholine (DPPC).

According to another embodiment, the phosphatidylcholine lipid distearoylphosphatidylcholine (DSPC) or dioleoylphosphatidylcholine (DOPC).

In a further embodiment, the phosphatidylcholine content is between 38 mol % and 60 mol %.

In another embodiment, the phosphatidylcholine content is between 40 mol % and 60 mol %, between 42 mol % and 60 mol %, between 45 mol % and 60 mol %, between 46 mol % and 60 mol % or between 48 mol % and 60 mol %.

In another embodiment, the cationic lipid is an amino lipid.

According to a further embodiment, the ionizable, cationic lipid is present at less than 20 mol %.

In another embodiment, the lipid nanoparticle comprises a hydrophilic polymer-lipid conjugate that is present at a lipid content of 0 mol % to 5 mol % or 0.5 mol % to 5 mol %.

In a further embodiment, the sterol is present at from 15 mol % to 45 mol % based on the total lipid present in the lipid nanoparticle.

According to a further embodiment, the sterol is present at from 18 mol % to 40 mol % based on the total lipid present in the lipid nanoparticle.

According to a further embodiment, the lipid nanoparticle exhibits at least a 10% increase in biodistribution in the liver, spleen, bone marrow, heart, lungs, kidney, abdominal skin, back skin and/or ear as compared to a baseline formulation without the targeting moiety but otherwise identical and/or an Onpattro-type formulation encapsulating the nucleic acid, but otherwise measured under an identical set of conditions, and wherein the biodistribution is quantified in an animal model by detection of a labelled lipid at 24 hours post-administration.

In another embodiment, the lipid nanoparticle exhibits at least a 10% increase in mRNA expression in the liver, spleen, bone marrow, heart, lungs, kidney, abdominal skin, back skin and/or ear as compared to a baseline formulation without the targeting moiety but otherwise identical and/or an Onpattro-type formulation encapsulating the nucleic acid and wherein the biodistribution is quantified in an animal model by detection of a labelled lipid at 24 hours post-administration.

According to a further embodiment, the lipid nanoparticle exhibits at least a 10% increase in biodistribution in the spleen, bone marrow, heart, lungs, kidney, abdominal skin, back skin and/or ear as compared to a baseline formulation without the targeting moiety but otherwise identical and/or an Onpattro-type formulation encapsulating the nucleic acid, but otherwise measured under an identical set of conditions, and wherein the biodistribution is quantified in an animal model by detection of a labelled lipid at 24 hours post-administration.

In another embodiment, the lipid nanoparticle exhibits at least a 10% increase in mRNA expression in the spleen, bone marrow, heart, lungs, kidney, abdominal skin, back skin and/or ear as compared to a baseline formulation without the targeting moiety but otherwise identical and/or an Onpattro-type formulation encapsulating the nucleic acid and wherein the biodistribution is quantified in an animal model by detection of a labelled lipid at 24 hours post-administration.

According to another embodiment, the targeting moiety is present at less than 2 mol %.

In a further embodiment, the targeting moiety is present at less than 1.8 mol %.

According to a further embodiment, the targeting moiety is present at less than 1.5 mol %.

In a further embodiment, the targeting moiety is present at less than 1.2 mol %.

According to a further aspect, there is provided a method for delivering a nucleic acid to a cell to treat a disease, disorder or condition, the method comprising contacting the lipid nanoparticle of any one of the foregoing aspects or embodiments with the cell in vivo or in vitro.

According to one embodiment, the nucleic acid accumulates in the spleen, bone marrow, heart, lungs and/or kidney of the subject at least one day post-administration.

In one embodiment, the disease, disorder or condition is an autoimmune disorder.

In another embodiment, the disease, disorder or condition is an infectious disease.

In one embodiment, the disease, disorder or condition is cancer.

In a further embodiment, the cell is a stem cell.

In one embodiment, the stem cell is a hematopoietic stem or progenitor cell.

In a further embodiment, the cell is a T-cell.

In another aspect, there is provided a use of the lipid nanoparticle described in any aspect or embodiment above for in vivo or in vitro delivery of the nucleic acid to mammalian cells.

In another aspect, there is provided a use of the lipid nanoparticle described in any aspect or embodiment above for the manufacture of a medicament for in vivo or in vitro delivery of the nucleic acid to mammalian cells.

### BRIEF DESCRIPTION OF THE FIGURES

FIG. 1 shows particle size, encapsulation efficiency and polydispersity index (PDI) of example lipid nanoparticles (LNPs) of the disclosure surface modified with different amounts of an arginine-glycine-aspartate (RGD) peptide targeting moiety linked to a lipid via PEG (LNPs B and C) vs a non-targeted control (LNP A). The LNPs A-C examined are described in Table 1 in Example 1 and encapsulate mRNA encoding firefly luciferase (Fluc).

FIG. 2 shows luminescence intensity per μg protein at various doses of example lipid nanoparticles (LNPs) of the disclosure surface modified with an arginine-glycine-aspartate (RGD) peptide targeting moiety linked to a lipid via PEG (LNP E; squares) vs a non-targeted control (LNP D; circles). The LNPs were added to A7 Astrocyte cell lines at the doses of mRNA indicated. LNPs D and E examined are described in Table 2 in Example 2.

FIG. 3A shows luminescence intensity in the liver of example lipid nanoparticles (LNPs) of the disclosure surface modified with different amounts of an arginine-glycine-aspartate (RGD) peptide targeting moiety linked to a lipid (LNPs B and C) via PEG vs a non-targeted control (LNP A). The LNPs A-C examined are described in Table 1 in Example 1 and encapsulate mRNA encoding firefly luciferase (Fluc).

FIG. 3B shows luminescence intensity in the bone marrow of example lipid nanoparticles (LNPs) of the disclosure surface modified with different amounts of an arginine-glycine-aspartate (RGD) peptide targeting moiety linked to a lipid via PEG (LNPs B and C). The LNPs B and C examined are described in Table 1 in Example 1 and encapsulate mRNA encoding firefly luciferase (Fluc).

FIG. 4A shows enhanced green fluorescent protein (GFP) in Lineage⁻c-Kit⁺ (LK) and Lineage⁻c-Kit⁺Scal⁺ (LSK) cell populations in the bone marrow after injection of phosphate buffered saline (PBS), an Onpattro™-type formulation (B), an lcLNP™ having no ligand (C), an lcLNP™ with an aCD117 ligand (D) and lcLNP™ with aCD5 ligand (E) to mice. The formulations are set out in Table 3 and the gating schemes in Table 4.

FIG. 4B shows enhanced green fluorescent protein (GFP) in multipotent progenitors Lineage⁻ckit⁺Scal⁺CD34⁺ (MPP), short term HSC Lineage⁻ckit⁺Scal⁺CD34-CD135⁺ (ST-HSC) and Lineage⁻ckit⁺Scal⁺CD34⁻CD135⁻ (LT-HSCs) cell populations after injection of phosphate buffered saline (PBS), an Onpattro™-type formulation (B), an lcLNP™ having no ligand (C), an lcLNP™ with an aCD117 ligand (D) and lcLNP™ with aCD5 ligand (E) to mice. The formulations are set out in Table 3 and the gating schemes in Table 4.

FIG. 4C shows enhanced green fluorescent protein (GFP) in Lineage ckit⁺Scal⁺CD34⁻CD135⁻CD48⁻CD150⁺ cell populations after injection of phosphate buffered saline (PBS), an Onpattro™-type formulation (B), an lcLNP™ having no ligand (C), an lcLNP™ with an aCD117 ligand (D) and lcLNP™ with aCD5 ligand (E) to mice. The formulations are set out in Table 3 and the gating schemes in Table 4.

FIG. 5 is a cryo-TEM image of a lipid nanoparticle composed of 50 mol % DSPC, namely MF019/DSPC/Chol/PEG-DMG (27.4/50/21.1/1.5 mol:mol) encapsulating mRNA encoding luciferase. MF019 is an ionizable, cationic lipid disclosed in WO 2022/155728A1, which is incorporated herein by reference.

### DETAILED DESCRIPTION

The lipid nanoparticles described herein comprise a targeting moiety together with ionizable lipid, elevated levels of neutral lipid, such as a phosphatidylcholine lipid (e.g., DSPC) or a sphingolipid (e.g., sphingomyelin lipid), or the like and a sterol. In certain embodiments, the neutral lipid is a phosphatidylcholine lipid that is present at a mol % of at least 35 mol %, at least 40 mol % or at least 42 mol % and in which the ionizable lipid is present at less than 45 mol % or 40 mol %. At set forth herein, in certain non-limiting examples of the disclosure, the inclusion of neutral lipids at a mol % higher than that used in conventional formulations for nucleic acid delivery, in combination with a targeting ligand, provides improvements in the selective delivery of nucleic acid to hepatic and/or extrahepatic tissues relative to Onpattro™-type LNP or an identical LNP that does not include the targeting moiety.

### Neutral Lipid

The neutral lipid is an amphipathic lipid that allows for the formation of particles and has substantially no net charge at physiological pH. The term includes zwitterionic lipids, such as but not limited to phospholipids. In alternative embodiments, the lipid nanoparticle comprises a structural lipid is a non-cationic lipid. In certain embodiments, the LNP has substantially no net charge.

As used herein “substantially no net charge”, with reference to an LNP means a net surface charge of about zero, or near neutral at physiological pH, such as without limitation about-2.5 mV to about 2.5 mV.

In some embodiments, the neutral lipid is a phosphatidycholine lipid. The phosphatidylcholine lipid may be selected from distearoylphosphatidylcholine (DSPC), dioleoylphosphatidylcholine (DOPC), 1-palmitoyl-2-oleoyl-phosphatidylcholine (POPC), dimyristoylphosphatidylcholine (DMPC) and dipalmitoyl-phosphatidylcholine (DPPC). In another embodiment, the phosphatidylcholine lipid may be selected from distearoylphosphatidylcholine (DSPC), dioleoylphosphatidylcholine (DOPC) and dipalmitoyl-phosphatidylcholine (DPPC) and mixtures thereof. The phosphatidylcholine lipid component may include mixtures of two or more types of different neutral lipids.

The phosphatidylcholine content in some embodiments is greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of neutral lipid content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the phosphatidylcholine lipid content is from 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle.

In some embodiments, the lipid nanoparticle comprises 35 to 60 mol % or 35 to 55 mol % of one of distearoylphosphatidylcholine (DSPC), dioleoylphosphatidylcholine (DOPC) or dipalmitoyl-phosphatidylcholine (DPPC). In some embodiments, the lipid nanoparticle comprises 40 to 60 mol % or 40 to 55 mol % of one of distearoylphosphatidylcholine (DSPC), dioleoylphosphatidylcholine (DOPC) or dipalmitoyl-phosphatidylcholine (DPPC).

In one embodiment, most advantageously, the neutral lipid is DSPC. In some examples of the disclosure, the DSPC lipid present at elevated levels relative to an Onpattro™-type LNP improves the biodistribution of the LNP over other neutral phospholipids.

The term “Onpattro™-type” with reference to an LNP that is compared to an LNP of the present disclosure is an LNP having ionizable lipid/DSPC/chol/PEG2000-DMG lipid at 50/10/38.5/1.5 mol/mol, wherein the ionizable lipid is the same as the ionizable lipid of the LNP being assessed.

In certain embodiments, the DSPC lipid content is from 20 mol % to 80 mol % or 25 mol % to 60 mol % or 30 mol % to 60 mol % or 35 mol % to 60 mol % or 38 mol % to 60 mol % or 40 mol % to 60 mol % or 42 mol % to 60 mol %, or 43 mol % to 60 mol % or 44 mol % to 60 mol % or 45 mol % to 60 mol % or 46 mol % to 60 mol % or 48 mol % to 60 mol % of total lipid present in the lipid nanoparticle. In certain embodiments, the DSPC lipid content is from 30 mol % to 55 mol % or 35 mol % to 55 mol % or 38 mol % to 55 mol % or 40 mol % to 55 mol % or 42 mol % to 55 mol %, or 43 mol % to 55 mol % or 44 mol % to 55 mol % or 45 mol % to 55 mol % of total lipid present in the lipid nanoparticle.

In one embodiment, the neutral lipid is DOPC. In some examples of the disclosure, the DOPC lipid at elevated improves the biodistribution of the LNP over other neutral phospholipids.

In certain embodiments, the DOPC lipid content is from 20 mol % to 80 mol % or 25 mol % to 60 mol % or 30 mol % to 60 mol % or 35 mol % to 60 mol % or 40 mol % to 60 mol % or 42 mol % to 60 mol %, or 43 mol % to 60 mol % or 44 mol % to 60 mol % or 45 mol % to 60 mol % or 46 mol % to 60 mol % or 48 mol % to 60 mol % of total lipid present in the lipid nanoparticle.

In one embodiment, the neutral lipid is DPPC. In some examples of the disclosure, the DPPC lipid at elevated improves the biodistribution of the LNP over other neutral phospholipids. In certain embodiments, the DPPC lipid content is from 20 mol % to 80 mol % or 25 mol % to 60 mol % or 30 mol % to 60 mol % or 35 mol % to 60 mol % or 40 mol % to 60 mol % or 42 mol % to 60 mol %, or 43 mol % to 60 mol % or 44 mol % to 60 mol % or 45 mol % to 60 mol % or 46 mol % to 60 mol % or 48 mol % to 60 mol % of total lipid present in the lipid nanoparticle.

The neutral lipid may also include sphingolipid, such as a ceramide, a sphingomyelin, a cerebroside, a ganglioside, or derivatives, such as but not limited to reduced analogues thereof, that lack a double bond in the sphingosine unit. In some embodiments, the sphingolipid is present at 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle. In some embodiments, the sphingomyelin is present at 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle.

The sphingomyelin content of the lipid nanoparticle in some examples is less than 5 mol %, less than 4 mol %, less than 3 mol %, less than 2 mol %, less than 1 mol %, less than 0.75 mol %, or less than 0.5 mol %. In some embodiments, the LNP is “sphingomyelin-free”, meaning there is no detectable sphingomyelin in the LNP (less than 0.5 mol %) or the LNP is substantially sphingomyelin-free, meaning there is less than 5 mol % sphingomyelin in the LNP.

The LNP may comprise additional neutral lipids besides a phosphatidylcholine lipid. For example, the LNP may comprise other lipids that have a net positive or negative charge at physiological pH. In another example, the LNP may further comprise lesser amounts of one or more fusogenic lipids (relative to the phosphatidylcholine lipids), such as DOPE, which are cone-shaped and thereby promote fusion with a cell membrane. Generally, such non-phosphatidylcholine lipids will be present in the LNP at less than 10 mol %, less than 9 mol %, less than 8 mol %, less than 7 mol %, less than 6 mol % or less than 5 mol % relative to total lipid present in the LNP.

The inclusion of fusogenic lipids, such as dioleoylphosphatidylethanolamine (DOPE) is thought to facilitate nucleic acid delivery in vitro or in vivo. However, the present disclosure generally does not favour the inclusion of such lipids. Accordingly, the fusogenic lipid content of the lipid nanoparticle in some examples is less than 5 mol %, less than 4 mol %, less than 3 mol %, less than 2 mol %, less than 1 mol %, less than 0.75 mol %, or less than 0.5 mol %. In some embodiments, the LNP is “fusogenic lipid-free”, meaning there are no detectable amounts of fusogenic lipids present in the LNP (less than 0.5 mol %) or the LNP is substantially fusogenic lipid-free, meaning there is less than 5 mol % fusogenic lipid content measured relative to total lipid content in the LNP.

The DOPE content of the lipid nanoparticle in some examples is less than 10 mol %, less than 8 mol %, 5 mol %, less than 4 mol %, less than 3 mol %, less than 2 mol %, less than 1 mol %, less than 0.75 mol %, or less than 0.5 mol %. In some embodiments, the LNP is “DOPE-free”, meaning there is no detectable DOPE in the LNP (less than 0.5 mol %) or the LNP is substantially DOPE-free, meaning there is less than 5 mol % DOPE measured relative to total lipid content in the LNP.

In further embodiments, it might be advantageous to include mixtures of different phosphatidylcholine lipids in the LNP. However, in some examples, the phosphatidylcholine lipid content includes less than 5, 4, or 3 different phosphatidylcholine lipids.

The egg phosphatidylcholine (EPC) content of the lipid nanoparticle in some examples is less than 5 mol %, less than 4 mol %, less than 3 mol %, less than 2 mol %, less than 1 mol %, less than 0.75 mol %, or less than 0.5 mol %. In some embodiments, the LNP is “EPC-free”, meaning there is no detectable EPC (less than 0.5 mol %) in the LNP or the LNP is substantially EPC-free, meaning there is less than 5 mol % EPC in the lipid nanoparticle measured relative to total lipid content in the LNP.

In another embodiment the structural, neutral, zwitterionic or non-cationic lipid content of the lipid nanoparticle is composed of less than 20, 10, or 5 mol % of non-phosphatidylcholine lipids, such as POPC (measured relative to total phosphatidylcholine, structural lipid or neutral lipid content).

In some embodiments, the transition temperature of the structural, neutral, zwitterionic or non-cationic lipid, e.g., a phospholipid having a choline head group, is at least 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. or 38° C. A hydrophilic polymer-lipid conjugate is often included in LNPs to avoid fusion and agglomeration of the particles. Without intending to be limited by any particular theory, it is believed that fusion and agglomeration of lipid nanoparticles with no hydrophilic polymer lipid conjugate (or low levels thereof) during particle formation using the mixing method described in co-owned and co-pending U.S. provisional patent application No. 63/588,167 filed on Oct. 5, 2023, which is incorporated herein by reference, could be avoided by selecting a structural, neutral, zwitterionic or non-cationic lipid that is in the gel phase rather than in the disordered liquid crystalline phase at room temperature and above. The inclusion of such structural, neutral, zwitterionic or non-cationic lipid in the lipid nanoparticle may also improve blood stability after injection.

In one embodiment, the phase transition temperature of the neutral lipid, or mixture thereof, when incorporated in the lipid nanoparticle is at least 38, 39 or 40 degrees Celsius.

The neutral lipid content is determined based on the total amount of lipid in the lipid nanoparticle, including the sterol (mol:mol).

### Ionizable Lipid

The LNP of the disclosure comprises an ionizable lipid. The ionizable lipid may be charged at low pH and have substantially no net charge at physiological pH. This allows for electrostatic interactions between the lipid and the negatively charged nucleic acid cargo during initial formulation. Since the ionizable lipid is near neutral at physiological pH, toxicity and renal clearance is reduced. Without being limited by theory, after cellular uptake by endocytosis, the acidic environment of the endosome leads to an increase in the net positive charge of the ionizable amino lipids, which promotes fusion with the anionic lipids of the endosomal membrane and subsequent membrane destabilization and release of the nucleic acid-based therapeutics into the cytoplasm to exert their effects.

In some embodiments, the LNP has an apparent pKa of between 5.0 and 8.5, between 5.0 and 8.0, between 5.0 and 7.5, between 6.5 and 7.5 or between 6.6 and 7.3. The apparent pKa is measured using a 6-(p-Toluidino)-2-naphthalenesulfonic acid (TNS) assay adapted from previous studies from other groups (Shobaki et al., 2018, International Journal of Nanomedicine, 13:8395-8410; and Jayaraman et al., 2012, Angew. Chem Int. Ed., 51:8529-8533, which are incorporated herein by reference for the purposes of determining apparent pKa). According to the method, a series of buffers are prepared spanning a pH range of 2-11 in 0.5 pH unit increments consisting of 130 mM NaCl, 10 mM ammonium acetate, 10 mM 2-(N-morpholino) ethanesulfonic acid (MES), and 10 mM HEPES. 0.15-0.2 mM of the LNP. A solution of 0.06 mM of TNS is subsequently mixed with 175 μL of the LNP at each buffered pH in triplicate in a black, polysterene 96-well plate, to yield a final concentration of 6.25 and 6 μM of lipid and TNS in each well, respectively. Fluorescence is subsequently measured using an SpectraMax™ M5 microplate reader at Alex=321 nm, λₑₘ=445 nm. The fluorescence is then plotted against pH using a sigmoidal curve fit through Prism™, in which the pKa is determined to be the pH value with 50% of maximal fluorescent intensity.

In some embodiments, it is desirable to include less than 50 mol % ionizable lipid in the LNP. That is, the ionizable lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol % as measured based on total lipid content of the LNP.

In certain embodiments, the ionizable lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

The ionizable lipid may be referred to as a “cationic lipid”. As used herein, the term “cationic lipid” refers to a lipid that, at a given pH, such as physiological pH, is in an electrostatically neutral form and that accepts protons at a lower pH, thereby becoming electrostatically positively charged, and for which the electrostatically neutral form has a calculated logarithm of the partition coefficient between water and 1-octanol (i.e., a cLogP) greater than 8. In some embodiments, the cationic lipid has a pKa that is between 5.0 and 7.0.

In some embodiments, the cationic lipid has an amino group. In some embodiments, the cationic lipid comprises a protonatable tertiary amine (e.g., pH titratable) head group and two alkyl chains having 0 to 3 double bonds. Such lipids include, but are not limited to sulfur lipids, such as MF019 described herein and DODMA. Other lipids that may be used in the practice of the disclosure include MC3- and KC2-type lipids, which are well-known to those of skill in the art. In further embodiments, the ionizable lipid is selected from one or more lipids set forth in WO 2022/246555; WO 2022/246568; WO 2022/246571; WO 2023/147657; WO 2022/155728; WO 2023/215989; PCT/CA2023/051272 filed on Sep. 27, 2023; PCT/CA2023/051273 filed on Sep. 27, 2023; U.S. provisional patent application No. 63/434,506 filed on Dec. 22, 2022; PCT/CA2023/051274 filed on Sep. 27, 2023; and U.S. provisional patent application No. 63/445,854 filed on Feb. 15, 2023, each incorporated herein by reference.

In one embodiment, the ionizable cationic lipid has a protonatable amino head group; at least two lipophilic moieties, wherein the amino head group has a central nitrogen atom or carbon atom to which each of the two lipophilic moieties are directly bonded; each lipophilic chain has between 15 and 40 carbon atoms in total; and wherein the lipid has (i) a pKa of between 6 and 8.0 (e.g., when formulated); and (ii) a logP of at least 11.

Optionally, at least one of the lipophilic moieties bonded to the head group has a biodegradable group. In one non-limiting example, at least one of the lipophilic moieties has an ester group in any orientation and a sulfur atom (e.g., see U.S. provisional patent application No. 63/434,506 filed on Dec. 22, 2022, incorporated herein by reference). In one embodiment, the ionizable cationic lipid has at least one lipophilic moiety of the formula:

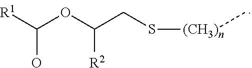

In one embodiment, R¹ and R² are, independently, linear, cyclic or branched optionally substituted C₃-C₂₀ alkyl and optionally with varying degrees of unsaturation; and n is 2 to 8 or 4 to 8.

In some embodiments, it is desirable to include less than 50 mol % cationic lipid in the LNP. That is, the ionizable lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol %.

In certain embodiments, the cationic lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

The inclusion of permanently positively charged lipids, such as dimethyldioctadecylammonium bromide (DDAB), 1,2-di-O-octadecenyl-3-trimethylammonium propane (DOTMA), 1,2-dioleoyl-3-trimethylammoniumpropane (DOTAP), 2,3-dioleyloxy-N-[2-(sperminecarboxamido)ethyl]-N,N-dimethyl-1-propanaminium (DOSPA) and cholesterol-imidazolium (CHIM) in LNPs is thought to facilitate nucleic acid transfection in vitro or in vivo. Such permanently charged lipids have a quarternary amine that is not ionizable and thus is permanently charged. However, the present disclosure generally does not favour the inclusion of such permanently charged lipids. Accordingly, the permanently positively charged lipid content of the lipid nanoparticle in some examples is less than 5 mol %, less than 4 mol %, less than 3 mol %, less than 2 mol %, less than 1 mol %, less than 0.75 mol %, or less than 0.5 mol %. In some embodiments, the LNP is “free of permanently charged cationic lipid”, meaning there are no detectable amounts of permanently charged cationic lipids present in the LNP (less than 0.5 mol %) or the LNP is substantially free of permanently charged cationic lipid, meaning there is less than 5 mol % or less than 3 mol % of permanently charged cationic lipid content measured relative to total lipid content in the LNP.

The DDAB content of the lipid nanoparticle in some examples is less than 10 mol %, less than 8 mol %, 5 mol %, less than 4 mol %, less than 3 mol %, less than 2 mol %, less than 1 mol %, less than 0.75 mol %, or less than 0.5 mol %. In some embodiments, the LNP is “DDAB-free”, meaning there is no detectable DDAB in the LNP (less than 0.5 mol %) or the LNP is substantially DDAB-free, meaning there is less than 5 mol % DDAB measured relative to total lipid content in the LNP.

The DOTMA content of the lipid nanoparticle in some examples is less than 10 mol %, less than 8 mol %, 5 mol %, less than 4 mol %, less than 3 mol %, less than 2 mol %, less than 1 mol %, less than 0.75 mol %, or less than 0.5 mol %. In some embodiments, the LNP is “DOTMA-free”, meaning there is no detectable DOTMA in the LNP (less than 0.5 mol %) or the LNP is substantially DOTMA-free, meaning there is less than 5 mol % DOTMA measured relative to total lipid content in the LNP.

The DOTAP content of the lipid nanoparticle in some examples is less than 10 mol %, less than 8 mol %, 5 mol %, less than 4 mol %, less than 3 mol %, less than 2 mol %, less than 1 mol %, less than 0.75 mol %, or less than 0.5 mol %. In some embodiments, the LNP is “DOTAP-free”, meaning there is no detectable DOTAP in the LNP (less than 0.5 mol %) or the LNP is substantially DOTAP-free, meaning there is less than 5 mol % DOTAP measured relative to total lipid content in the LNP.

The DOSPA content of the lipid nanoparticle in some examples is less than 10 mol %, less than 8 mol %, less than 5 mol %, less than 4 mol %, less than 3 mol %, less than 2 mol %, less than 1 mol %, less than 0.75 mol %, or less than 0.5 mol %. In some embodiments, the LNP is “DOTAP-free”, meaning there is no detectable DOSPA in the LNP (less than 0.5 mol %) or the LNP is substantially DOSPA-free, meaning there is less than 5 mol % DOSPA measured relative to total lipid content in the LNP.

The CHIM content of the lipid nanoparticle in some examples is less than 10 mol %, less than 8 mol %, 5 mol %, less than 4 mol %, less than 3 mol %, less than 2 mol %, less than 1 mol %, less than 0.75 mol %, or less than 0.5 mol %. In some embodiments, the LNP is “CHIM-free”, meaning there is no detectable CHIM in the LNP (less than 0.5 mol %) or the LNP is substantially CHIM-free, meaning there is less than 5 mol % CHIM measured relative to total lipid content in the LNP.

The ionizable lipid component may include an ionizable anionic lipid as part of the ionizable lipid content. An example of such a lipid is cholesteryl hemisuccinate (CHEMS). Further examples of ionizable anionic lipids are described in co-pending and co-owned U.S. provisional patent application No. 63/453,766 titled “Ionizable Anionic Lipids” filed on Mar. 22, 2023, which is incorporated herein by reference in its entirety.

In some embodiments, the ionizable cationic lipid is not a lipidoid structure, including but not limited to C12-200 (see Khare et al., 2022, AAPS Journal, 24:8, incorporated by reference) and related structures known to those of skill in the art.

### Sterol

The term “sterol” refers to steroids that are naturally-occurring or synthetic. The term includes cholesterol, phytosterols, zoosterols and derivatives thereof.

The term “sterol derivatives” refers to modified sterols or precursors thereof, including triterpenes.

The term “cholesterol” refers to a naturally-occurring or synthetic compound having a gonane skeleton and that has a hydroxyl bonded to one of its rings, typically the A-ring.

The LNP may alternatively or additionally comprise a “cholesterol derivative”. The cholesterol derivative may be naturally-occurring or synthetic and includes but is not limited to a cholesterol molecule having a gonane structure and one or more additional functional groups, including derivatization of the terminal hydroxyl group.

The cholesterol derivative includes β-sitosterol, 3-sitosterol, campesterol, stigmasterol, fucosterol, or stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N—(N′N′-dimethylaminoethyl) carbamoyl cholesterol (DC-Chol), 24 (S)-hydroxycholesterol, 25-hydroxycholesterol, 25 (R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol or a salt or ester thereof.

In one embodiment, the sterol is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is cholesterol and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is a cholesterol derivative and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In one embodiment, the combined (i) sterol content (e.g., cholesterol or cholesterol derivative thereof); and (ii) phosphatidylcholine lipid content is at least 50 mol %; at least 55 mol %, at least 60 mol %, at least 65 mol %, at least 70 mol %, at least 75 mol %, at least 80 mol % or at least 85 mol % based on the total lipid present in the lipid nanoparticle.

In one embodiment, the sterol:ionizable lipid molar ratio is 0.70 to 1.30 or any range therebetween.

### Hydrophilic Polymer-Lipid Conjugate

In one non-limiting example, the lipid nanoparticle comprises a hydrophilic-polymer lipid conjugate capable of incorporation into the LNP. The conjugate includes a lipophilic moiety (e.g., lipid moiety) and a polymer chain that is hydrophilic, optionally with a linker (e.g., succinate) between the lipophilic moiety and the polymer chain. Examples of hydrophilic polymers include polyethyleneglycol (PEG), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic-polymer lipid conjugate is a PEG-lipid conjugate. The hydrophilic polymer lipid conjugate may also be a naturally-occurring or synthesized oligosaccharide-containing molecule, such as monosialoganglioside (G_{M1}). The ability of a given hydrophilic-polymer lipid conjugate to enhance the circulation longevity of the LNPs herein could be readily determined by those of skill in the art using known methodologies.

The hydrophilic polymer lipid conjugate may be present in the nanoparticle at 0.5 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid.

In certain embodiments, the hydrophilic polymer lipid conjugate may be absent or present in the nanoparticle. For example, the hydrophilic polymer-lipid conjugate may be presentat 0 mol % to 5 mol %, or at 0 mol % to 3 mol %, or at 0 mol % to 2.5 mol % or at 0 mol % to 2.0 mol % or at 0 mol % to 1.8 mol % of total lipid.

In another embodiment, the PEG-lipid conjugate is present in the nanoparticle at 0.5 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In one embodiment, the hydrophilic polymer-lipid is selected based on its exchangeability from the lipid nanoparticles. Such property may facilitate in vivo efficacy due to at least partial loss of the hydrophilic polymer-lipid conjugate from the LNP as it reaches a target site in vivo.

In such embodiment, the lipid moiety of the hydrophilic polymer-lipid conjugate typically has lipophilic chain lengths of less than 18 carbon atoms and having 0-2 double bonds in one or both of the chains. In some embodiments, the hydrophilic polymer-lipid conjugate is a PEG-lipid conjugate selected from dimyristoylphosphatidylethanolamine-PEG (DMPE-PEG), dipalmitoylphosphatidylethanolamine-PEG (DPPE-PEG), dioleylphosphatidylethanolamine-PEG (DOPE-PEG), dipalmitoylphosphatidylethanolamine-PEG (DPPE-PEG), dimyristoyldiglyceride-PEG (DMG-PEG) or cholesterol-PEG (Chol-PEG).

In one embodiment, the hydrophilic polymer lipid conjugate is not DSPE-PEG. In further embodiments, the DSPE-PEG content is less than 0.5 mol %, 0.45 mol %, 0.40 mol %, 0.35 mol %, 0.30 mol %, 0.25 mol %, 0.20 mol % or 0.15 mol %.

In a further embodiment, within the hydrophilic polymer lipid conjugate a cleavable linker is present between the lipid moiety and the hydrophilic polymer. Such linkers may be cleavable by exposure to low pH, reducing agents or proteases present in vivo. Examples of cleavable linkers include esters, ethers, phosphoroamidate, hydrazone, beta-thiopropionate, disulfide groups and peptides (Romberg et al., 2008, Pharmaceutical Research, 25:55-71, incorporated herein by reference).

### Additional Lipid Components

The LNP may comprise additional lipid components besides those described above (neutral lipid, cholesterol, ionizable cationic lipid and the optional hydrophilic polymer-lipid conjugate). Without limitation, such additional lipid components may be present at less than 10 mol %, 9 mol %, 8 mol %, 7 mol %, 6 mol %, 5 mol %, 4 mol %, 3 mol %, 2 mol %, 1 mol % or 0.5 mol % (relative to total lipid in the LNP). Such additional lipids include lipids comprising a targeting moiety, charged lipid (cationic or anionic lipid that is charged at physiological pH) or other lipid components such as vitamins (e.g., tocopherol). In some embodiments, the LNP consists essentially of neutral lipid, cholesterol, ionizable cationic lipid and the optional hydrophilic polymer-lipid conjugate, meaning any additional lipid is present at less than 5 mol % measured relative to total lipid in the LNP.

In one embodiment, the LNP lacks a ligand-lipid conjugate for targeting to stem or progenitor cells. In such embodiments, the ligand-lipid conjugate is undesirable as it may induce an immune response. Instead, targeting may be achieved by the inherent long circulating properties of the lcLNP™ due to elevated phosphatidylcholine content. Thus, in some embodiments the ligand-lipid conjugate is present at less than 1 mol %, less than 0.5 mol % or is 0 mol %.

The additional component may include an anionic phospholipid, such as phosphatidylserine, and/or an ionizable anionic lipid. An example of such a lipid is cholesteryl hemisuccinate (CHEMS). Further examples of ionizable anionic lipids are described in co-pending and co-owned U.S. provisional patent titled “Ionizable Anionic Lipids” filed on Mar. 23, 2023, which is incorporated herein by reference in its entirety.

Alternatively or additionally, the additional lipid component may include permanently charge cationic lipid, including lipids with a quarternary ammonium cation (e.g., DOTMA, DOSPA, DDAB, CHIM and DOTAP) or permanently charged anionic lipid, such as phosphatidylserine. Such permanently charged lipids, in some examples, are most advantageously present at less than 10 mol %, 9 mol %, 8 mol %, 7 mol %, 6 mol %, 5 mol %, 4 mol %, 3 mol %, 2 mol %, 1 mol %, 0.5 mol % or 0.25 mol % relative to total lipid content.

### Nanoparticle Preparation and Morphology

Delivery vehicles incorporating the nucleic acid can be prepared using a variety of suitable methods, such as a rapid mixing/ethanol dilution process. Examples of preparation methods are described in Jeffs, L. B., et al., Pharm Res, 2005, 22 (3): 362-72; and Leung, A. K., et al., The Journal of Physical Chemistry. C, Nanomaterials and Interfaces, 2012, 116 (34): 18440-18450, each of which is incorporated herein by reference in its entirety.

Without being bound by theory, the mechanism whereby a lipid nanoparticle comprising encapsulated nucleic acid can be formed using the rapid mixing/ethanol dilution process may be hypothesized as beginning with formation of a dense region of hydrophobic nucleic acid-ionizable lipid core at low pH (e.g., pH 4) surrounded by a monolayer of neutral lipid/sterol (e.g., cholesterol) that fuses with smaller empty vesicles as the pH is raised due to the conversion of the ionizable cationic lipid to the neutral form. As the proportion of bilayer neutral lipid increases, the bilayer lipid progressively forms blebs and the ionizable lipid migrates to the interior hydrophobic core. At high enough neutral lipid contents, the exterior bilayer preferring neutral lipid can form a complete bilayer around the interior trapped volume.

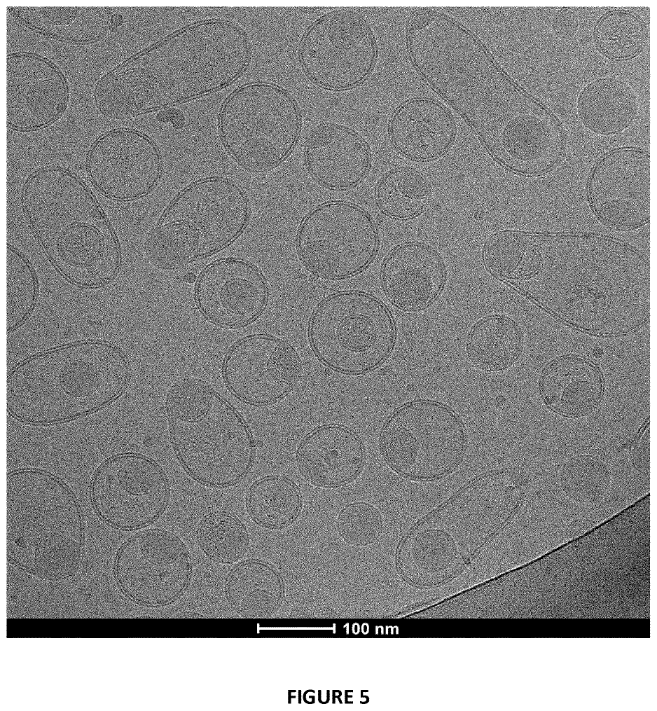

The LNP may comprise a “core” region. Surprisingly, it has been observed that the core is non-homogeneous in that it includes both an electron dense region and an aqueous portion or compartment as visualized by cryo-EM microscopy. Without being limiting, the electron dense region within the core may be partially surrounded by the aqueous compartment within the enclosed space as observed by cryo-TEM. The aqueous portion forms a distinct aqueous region or compartment within the lipid nanoparticle. In other words, the aqueous portion in some embodiments is not merely a hydration layer. In addition, such particles are described in co-owned and co-pending WO 2023/184038, the contents of which are incorporated herein by reference. FIG. 5 herein is a reproduction of LNPs having the electron dense region and aqueous portion from FIG. 16 of co-owned and co-pending WO 2023/184038.

In one embodiment, at least one about fifth of the core (trapped volume) contains the aqueous compartment, and in which the electron dense region is partially contiguous with the lipid layer comprising the bilayer, as determined qualitatively by cryo-EM. In another embodiment, at least one about quarter of the core contains the aqueous compartment, and in which the electron dense core is partially contiguous with the lipid layer comprising the bilayer, as determined qualitatively by cryo-EM. In a further embodiment, at least one about one third of the core contains the aqueous compartment, and in which the electron dense region is partially contiguous with the lipid layer comprising the bilayer, as determined qualitatively by cryo-EM. In another embodiment, at least one about one half of the core contains the aqueous compartment, and in which the electron dense core is partially contiguous with the lipid layer comprising the bilayer, as determined qualitatively by cryo-EM.

In one embodiment, the electron dense region is generally spherical in shape. In another embodiment, the electron dense region is hydrophobic.

In another embodiment, the electron dense region of the LNP surprisingly appears to be completely surrounded by the aqueous portion as visualized by cryo-TEM microscopy. This morphology is observed in a single plane and a portion of the electron dense region is contiguous with the bilayer but is not visualizable since this portion is not within the plane being visualized.

The lipid nanoparticle may comprise a single bilayer or comprise multiple lipid layers (i.e., multi-lamellar). The one or more lipid layers, including the bilayer, may form a continuous layer surrounding the core or may be discontinuous. The lipid layer may be a combination of a bilayer and a monolayer in some embodiments. In one non-limiting example, the lipid layer is a continuous bilayer that surrounds the core.

Thus, in certain embodiments the electron dense region of the core is separated from the lipid layer comprising the bilayer by the aqueous portion or compartment. For example, the disclosure provides a lipid nanoparticle preparation comprising a plurality of lipid nanoparticles in which at least 10%, 20%, 30%, 40%, 50%, 60% or 70% of the particles as determined by cryo-EM microscopy have a core with an electron dense region and an aqueous portion and in which the aqueous portion is partially surrounded by the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have an elongate shape (e.g., generally oval-shaped) as determined qualitatively by cryo-EM microscopy. In this latter embodiment, the electron dense region of the core may be partially surrounded by the aqueous space as visualized by cryo-EM microscopy.

In one embodiment, the lipid nanoparticle is part of a preparation of lipid nanoparticles, and wherein the electron dense region of at least 20% of the lipid nanoparticles are either (i) enveloped by the aqueous portion, or (ii) is partially surrounded by the aqueous portion and wherein a portion of a periphery of the electron dense region is contiguous with the lipid layer, as visualized by cryo-EM microscopy in a single plane.

In certain embodiments, the disclosure provides a lipid nanoparticle preparation comprising a plurality of lipid nanoparticles in which generally at least 10%, 20%, 30%, 40%, 50%, 60% or 70% of the particles as determined by cryo-EM microscopy have a core with an electron dense region that is contiguous with the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have a core comprising an electron dense region that appears to be surrounded or enveloped by a continuous aqueous space disposed between the lipid layer (e.g., bilayer) and the electron dense region, as visualized in one plane by cryo-EM microscopy.

LNPs are visualized by cryo-TEM as described in the Materials and Methods of the Example section.

In another embodiment, the polydispersity index (PDI) of the LNP preparation is less than 0.2, 0.15, 0.12 or 0.10.

In another embodiment, the particle size distribution is such that at least 90% of the particles in the LNP preparation of the disclosure have a diameter of between 40 and 150 nm or between 40 and 140 nm or between 45 and 150 nm or between 50 and 150 nm or between 50 and 120 nm or between 50 and 140 nm.

The lipid nanoparticles herein may exhibit particularly high encapsulation efficiencies of nucleic acid. As used herein, the term “encapsulation,” with reference to incorporating the nucleic acid within a lipid nanoparticle refers to any association of the nucleic acid with any lipid component or compartment of the lipid nanoparticle, including a lipophilic or the aqueous portion. In one embodiment, the nucleic acid is present at least in the core of the LNP.

In one embodiment, the encapsulation efficiency is at least 50, 55, 60, 65, 70, 75, 80, 85, 90% or 92%. The encapsulation efficiency of the nucleic acid is determined as set forth in the Materials and Methods section in the Examples herein.

Embodiments of the present disclosure also provide lipid nanoparticles described according to the molar ratio between the positively charged amine groups of the amine lipid (N) and the negatively charged phosphate groups (P) of the oligonucleotide to be encapsulated. This may be mathematically represented by the equation N/P. In one embodiment, the N/P ratio of the lipid nanoparticle is between 3 and 15, 4 and 15 or between 4.5 and 10 or between 5 and 10 or between 5.5 and 8.

In one embodiment, the N/P ratio of the lipid nanoparticle is at least 3, 3.25, 3.50, 3.75, 4, 4.25, 4.50, 4.75, 5.0, 5.25, 5.5, 5.75, 6.0 or 6.25. The upper limit may be 15, 14, 13, 12, 11, 10, 9 or 8. The disclosure also encompasses a combination of any two of the upper and lower limits.

In one embodiment, the lipid nanoparticle has a weight nucleic acid/micromole of total lipid that is 0.05:1 to 1:1. In one embodiment, the lower limit is 0.06:1, 0.08:1, 0.10:1, 0.12:1, 0.14:1, 0.16:1, 0.18:1, 0.20:1, 0.22:1, 0.24:1, 0.26:1, 0.28:1, 0.30:1, 0.32:1, 0.34:1, 0.36:1, 0.38:1 or 0.40:1 weight nucleic acid/micromole of total lipid. In another embodiment, the upper limit is 0.80:1, 0.82:1, 0.84:1, 0.86:1, 0.88:1, 0.90:1, 0.92:1, 0.94:1, 0.96:1 or 0.98:1 weight nucleic acid/micromole of total lipid. The disclosure also encompasses a combination of any two of the upper and lower limits.

In one embodiment, the mRNA copy number/LNP is 1-10 or 4-8.

### Targeting Moiety

The lipid nanoparticles contain associated therewith a targeting moiety that facilitates the binding to and entry of the LNP into a target cell by endocytosis. The targeting moiety is any molecule or fragment thereof on the LNP surface that binds a target cell, such as via a cell surface receptor or epitope present on the target cell. The targeting moieties in some examples are selected to recognize certain sub-sets of cells, such as pathological cells, for example, malignant cells or infectious agents. In some embodiments, the targeting moiety is referred to as a ligand.

The binding affinity of the targeting moiety to the target cell may be detectable by any means known in the art, for example, by any standard in vitro assay such as ELISA, flow cytometry, immunocytochemistry, surface plasmon resonance and the like. Fragments of the targeting moiety are to be considered a targeting moiety as used herein and may be used in certain examples of the present disclosure (provided the fragment can bind to the appropriate cell surface epitope).

Examples of targeting moieties include antibodies, nanobodies, proteins including, without limitation, DARPins, and antibodies or fragments thereof, peptides, carbohydrates (e.g., monosaccharides and polysaccharides), aptamers, small molecules and the like. Non-limiting examples of targeting moieties are described in Friedl et al., 2021, Adv. Funct. Mater. 31:2103347, which is incorporated herein by reference.

Non-limiting binding pairs are antibody-antigen, nanobody-antigen, DARPin-receptor, hormone-receptor, enzyme-substrate, nutrient (e.g., vitamin)-transport protein, growth factor-growth factor receptor and carbohydrate-lectin.

In one embodiment, the targeting moieties are proteins and peptides comprising antigen-binding sequences of an immunoglobulin, such as an antibody or fragment thereof. In a further embodiment, the targeting moieties are antigen-binding antibody fragments lacking Fc sequences. Such targeting moieties are Fab fragments of an immunoglobulin, F (ab) 2 fragments of immunoglobulin, Fv antibody fragments, or single-chain Fv antibody fragments (scFv). These fragments can be enzymatically derived or produced recombinantly.

In another embodiment, the targeting moiety may be a nanobody, which is a heavy chain antibody having a VHH. Nanobodies may be desirable in certain examples of the disclosure as they may be easier to produce at scale than polyclonal antibodies and/or may have improved stability.

In one embodiment, the targeting moieties are those that form a binding pair with the tyrosine kinase growth factor receptors which are overexpressed on the cell surfaces in many tumours. Exemplary tyrosine kinase growth factors are VEGF receptor, FGF receptor, PDGF receptor, IGF receptor, EGF receptor, TGF-alpha receptor, TGF-beta receptor, HB-EGF receptor, ErbB2 receptor, ErbB3 receptor, and ErbB4 receptor. EGF receptor vIII and ErbB2 (HER2) receptors are especially preferred for cancer treatment using the lipid nanoparticles herein as these receptors are specific to cancerous cells, such as malignant cells. Alternatively, the targeting moieties are selected to recognize cells in need of genetic correction, or genetic alteration by introduction of a beneficial gene, such as: epithelial cells, endocrine cells in genetically deficient organisms, in vitro embryonic cells, germ cells, stem cells or reproductive cells.

A non-limiting example of a surface modified LNP for cancer treatment is anti-HER ScFv that targets HER2 expressed on breast cancer cells. Binding to HER2 extracellular domain may cause inhibition of its activity. In another embodiment, HER2 activity in breast cancer cells may be inhibited by using ankyrin repeat proteins (DARPins). According to such embodiment, LNPs may be modified with biparatopic antitumor DARPins (bipDARPins) having two binding moieties, which recognize two extracellular domains of HER2. Both targeting moieties may act to trap and stabilize an inactive conformation of HER2. This is particularly efficacious to promote apoptosis in HER2-dependent tumor cells. (See Stüber et al., 2021, Communications Biology 4 (762); incorporated hereby by reference).

In another example, the LNP can be surface modified to bind to T-cells in order to introduce nucleic acid cargo. The T-cell targeted in some examples includes a CD5+ or CD4+ T-cell. A non-limiting example is the delivery of nucleic acid encoding a chimeric antigen receptor to the T-cell. Such therapy produces chimeric antigen receptor T cells (i.e., CAR T cells) that have been genetically engineered to produce an artificial T cell receptor that is specific to a desired target antigen. The resultant CAR T cells can be used to target an antigen present on the surface of a sub-set of cell types. Upon binding to the surface antigen, the CAR T cells become activated and exert a desired therapeutic and/or prophylactic effect against a target cell in vivo. This may include destroying cells through stimulated cell proliferation, cytotoxicity and/or increasing the secretion of factors that can affect other cells, including without limitation, cytokines, interleukins and/or growth factors. Such CAR T therapy can be used to treat or prevent a variety of disease indications, including cancer, immunological disorders or cardiovascular conditions. For example, such approach can be used to treat cardiac injury by delivering mRNA encoding an antifibrotic CAR to T lymphocytes in vivo. The mRNA is formulated in an LNP modified with a targeting moiety for CD5. Such an approach can be used to produce antifibrotic CAR T cells in vivo. (Rurik et al., 2022, Science, 7:375(6576): 91-96, incorporated herein by reference). The use of targeted LNPs with improved biodistribution in hepatic or extrahepatic tissues/organs could be used to deliver mRNA encoding for the chimeric antigen receptor to a variety of target cells to treat a wide range of diseases or conditions.

Another non-limiting example of targeting sub-sets of cells using LNPs includes conjugating CD4 antibody to LNPs to specifically target CD4⁺ cells, including T cells. This type of LNP targeting can be used to introduce nucleic acid to T cells in vivo and can be used in immunotherapy, such as to treat HIV or other disease conditions. (Tombácz et al., 2021, Mol Ther, 29 (11): 3293-3304, incorporated herein by reference).

An antibody conjugated LNP may be targeted to receptors present on stem and progenitor cells, such as HSPCs. Examples are CD117, CD49d, CD44 and IL-6R receptors expressed on HSPCs. Thus, the LNP may include an anti-CD49d, CD44 and IL-6R antibody.

In some embodiments, the lipid nanoparticle comprises two or more different targeting moieties.

The ligand may be attached to the LNP by any suitable method available in the art. The attachment may be covalent or non-covalent, such as by adsorption or complex formation. The attachment preferably involves a lipophilic molecular moiety capable of conjugating to the ligand by forming a covalent or non-covalent bond. The lipophilic molecular moiety may be referred to as an “anchor”. An anchor partitions into lipophilic environments such as bilayers, and thereby attaches the ligand to the LNP. Methods of the ligand attachment via a lipophilic moiety are known in the art.

A particularly suitable mode of ligand attachment to the LNP is by using a ligand conjugated to a lipophilic anchor through an intermediate polymer linker, such as, without limitation, a hydrophilic polymer. Targeting moieties conjugated to lipophilic anchors, such as a lipid, via a hydrophilic polymer intermediate linker advantageously become stably associated with LNPs of the present disclosure.

A linker can vary in size depending on the ligand. In one embodiment, the linker size varies between 0.50 kDa to 20 kDa, 1 to 10 kDa or 1.5 to 8 kDa. A polymer may be functionalized with a terminal group that reacts selectively with a functional group on the ligand. Typically, the linker is polyethylene glycol (PEG), although other polymeric linkers of varying length known to those of skill in the art can used as well.

The targeting moiety can also be conjugated directly to a lipophilic moiety. For example, the targeting moiety may be a sugar group that is part of a glycolipid that is synthetic or that is naturally occurring. Thus, the term “conjugated” or “conjugate” as used to refer to a molecule comprising a targeting moiety and lipophilic anchor includes targeting moiety-lipid conjugates prepared by synthetic conjugation methods or that are naturally occurring moieties with lipophilic regions.

The targeting moiety conjugated to the lipophilic anchor (directly or via a linker) may be incorporated into a lipid nanoparticle by including the conjugated lipid in a lipid mixture used to prepare the lipid nanoparticle. For example, using the ethanol rapid mixing technique, a lipid conjugated with a targeting moiety directly (e.g., a glycolipid) or via a linker (e.g., a polymer such as PEG) may be added to the ethanolic lipid solution (comprising the lipid components, including ionizable lipid, phosphatidylcholine, sterol and hydrophilic polymer-lipid conjugate), which is mixed with a buffered solution of the nucleic acid to form LNP particles in a T-junction mixer, and subsequently treated to increase the pH of the external solution above the pKa of the ionizable lipid.

In another example, the LNP is modified with a targeting moiety conjugated to the lipophilic anchor after formulation using a post-insertion technique. Such a method may be utilized if the targeting moiety is sensitive to the conditions employed during formulation (e.g., a peptide or protein, such as an antibody), such as high ethanol concentrations used in the ethanol rapid mixing technique. In one exemplary embodiment, the LNP particle may be conjugated with a protein or peptide, such as an antibody, using a lipid-PEG-maleimide conjugate. Such lipid-PEG-maleimide conjugate may be linked to an antibody functionalized with N-succinimidyl S-acetylthioacetate via the sulfhydryl groups on the antibody. Other post-insertion techniques could be used in the practice of the invention to introduce a targeting moiety to the surface of an LNP that is sensitive to the formulation conditions.

In another example, the targeting moiety (linked directly or indirectly to the lipophilic moiety via the linker) is present at less than 3.0, 2.8, 2.6, 2.4, 2.2, 2.0, 1.9, 1.8, 1.7, 1.6, 1.5, 1.4, 1.3, 1.2, 1.1 or 1.0 mol % relative to the total lipid content of the LNP.

In another example, the targeting moiety (linked directly or indirectly to the lipophilic moiety via the linker) is present at between 0.25 to 3 mol %, 0.30 to 1.5 mol % or 0.35 to 1.25 mol %.

### Nucleic Acid Cargo

The lipid nanoparticles comprise a nucleic acid cargo. As used herein, the term “encapsulation,” with reference to incorporating the nucleic within a nanoparticle refers to any association of the nucleic acid with any component or compartment of the lipid nanoparticle. In one embodiment, the nucleic acid is incorporated in the core of the lipid nanoparticle (as visualized by cryo-EM). In another embodiment, the nucleic acid is incorporated between two closely apposed layers of lipid.

The nucleic acid includes without limitation an oligonucleotide, vector DNA or mRNA.

### Oligonucleotide Cargo

The oligonucleotide cargo includes interfering RNA and antisense oligonucleotides described in more detail hereinafter. The “oligonucleotide” or “oligonucleotide cargo” is a single-stranded or double-stranded RNA or DNA molecule and has a length of between 5 and 500 nucleotides. The term includes an antisense oligonucleotide (ASO) that is single stranded and generally 30 to 500 nucleotides in length or a shorter length, double stranded silencing RNA molecule (siRNA), which is 3 to 40 nucleotides in length.

### Short Interfering RNA

The oligonucleotide in one embodiment is a “short interfering RNA” or “siRNA”, which is an RNA molecule capable of reducing or inhibiting the expression of a target gene or nucleic acid sequence in a cell. In one embodiment, the short interfering RNA may mediate the degradation of a target mRNA as measured in vitro or in vivo. In such embodiment, the siRNA may function via base-pairing (when single-stranded) with complementary sequences of a target mRNA and induce mRNA cleavage.

The siRNA is double stranded and may be of a variety of lengths but is generally less than 35 nucleotides in length. In some embodiments, the siRNA has a length such as 1 to 35 nucleotides in length or 15 to 30 nucleotides in length or 20 to 25 nucleotides in length.

In those embodiments in which the siRNA reduces expression of a target gene or sequence by complementary base pairing and degradation of mRNA, the siRNA may have substantial or complete identity to the gene that encodes a target sequence, or may comprise a region of mismatch (i.e., a mismatch motif). The sequence of the siRNA can correspond to the full-length target sequence, or a subsequence thereof.

The double-stranded siRNA encapsulated in the LNP may include duplex RNA, such as double stranded small interfering RNA, asymmetrical interfering RNA (aiRNA) or pre-miRNA or a hybrid molecule comprising both RNA and DNA. In one embodiment, the double-stranded RNA is self-complementary. In such embodiments, the siRNA may form a stem loop or hairpin structure at one end.

The siRNA encompassed by embodiments of the disclosure may be used to inhibit expression of a wide range of target polynucleotides. The siRNA molecule targeting a specific polynucleotide for any therapeutic, prophylactic or diagnostic application may be readily prepared according to procedures known in the art. An siRNA target site may be selected and corresponding interfering RNAs may be chemically synthesized, created by in vitro transcription, or expressed from a vector or PCR product.

As noted, the siRNA described herein may comprise a “mismatch motif” or “mismatch region”, which refers to a portion of the siRNA sequence that does not have 100% complementarity to its target sequence. An siRNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The nucleotides of the siRNA may or may not be chemically modified. Examples of optional modifications include, but are not limited to, 2′-O-alkyl modifications such as 2′-O-Me or 2′-O-methoxyethyl modifications and 2′-halogen modifications such as 2′-fluoro modifications. In yet further embodiments, the siRNA comprises one, two, three, four, or more 2′-deoxy nucleotides, e.g., in the sense and/or antisense strand of the double-stranded region. Alternately or additionally, in some embodiments, the siRNA comprises phosphate backbone modifications.

Within an siRNA, the antisense strand and the sense strand may be designed such that when they form a duplex due to complementarity base pairing, they can anneal with no overhangs and thus form blunt ends at both ends of the duplex, or with an overhang at one or more of the 3′ end of the sense strand, the 3′ end the antisense strand, the 5′ end of the sense strand, and the 5′ end of the antisense strand. In some embodiments, there are no 5′ overhangs and there is no 3′ antisense overhang, but there is a 3′ sense overhang. In other aspects, there are no 5′ overhangs, but there is a 3′ antisense overhang and a 3′ sense overhang. The overhangs may comprise T or U nucleotides.

In some embodiments, the siRNA is covalently bound to one or more other moieties to form a conjugate. In some embodiments, the conjugates are selected based on their ability to facilitate delivery of the siRNA to an organism or into cells. An siRNA may be bound to a moiety at, for example, the 5′ end of the antisense strand, the 3′ end of the antisense strand, the 5′ end of the sense strand, the 3′ end of the sense strand, or to a nucleotide at a position that is not at the 3′ end or 5′ end of either strand.

Examples of conjugates include but are not limited to one or more of an antibody or fragments thereof, a peptide, an amino acid, an aptamer, a phosphate group, a cholesterol moiety, a lipid, a cell-penetrating peptide, a polymer, and a sugar group, which includes a sugar monomer, an oligosaccharide and modifications thereof. In one non-limiting example, the conjugate is N-Acetylgalactosamine (GalNAc).

### Anti-Sense Oligonucleotide (ASO)

The nucleic acid cargo in one embodiment is an “antisense oligonucleotide” or “ASO”, which is a single strand of nucleic acid (e.g., RNA or DNA) that binds to a target nucleic acid sequence by base pairing. The ASO may have substantial or complete identity to the gene that encodes a target sequence, or may comprise a region of mismatch (i.e., a mismatch motif). The sequence of the ASO can correspond to the full-length target sequence, or a subsequence thereof.

The ASO may reduce or inhibit the expression of a target gene or nucleic acid sequence in a cell via a variety of mechanisms, some of which are described below. In one embodiment, the ASO forms part of a gene editing complex for directing a nuclease to a target site for site-specific cleavage of DNA.

In those embodiments in which the ASO reduces expression of a target gene or sequence by complementary base pairing and degradation of mRNA, the ASO exerts its effects via base-pairing with complementary sequences of a target mRNA and induces mRNA cleavage. The ASO may prevent or reduce the translation of a complementary RNA strand by binding to the RNA. ASOs can be used to target a specific, complementary (coding or non-coding) RNA. If binding occurs, a target sequence can be degraded by the enzyme RNase H, which exists in the nucleus and/or cytoplasm of cells. In one embodiment, the ASO is a “gapmer” sequence that comprises 2-5 chemically modified nucleotides on each terminus blanking a central gap region of DNA (e.g., 8-10 base “gap”). The chemically modified nucleotides decrease degradation by nucleases and increase affinity of the ASO for the target sequence. The gap allows formation of a hybrid sequence that can be cleaved by RNase H. In addition, an oligonucleotide can be chemically modified using known methods to recruit RNase H.

In another embodiment, the ASO may bind a target mRNA and block gene expression. ASOs that function by blocking gene expression are known as “steric blockers” and block binding of the ribosome, thereby preventing or reducing translation of the target nucleotide sequence.

In a further embodiment, the ASO may modulate splicing of a pre-mRNA sequence. ASOs can be designed to target sequences within a pre-mRNA to affect splicing and increase the production of a desired isoform. The ASO can be used, for example, to remove a mutant exon, thereby restoring a proper reading frame and producing a more functional protein product.

In one embodiment, the ASO comprises from about 15 to about 500 nucleotides or from about 20 to about 300 nucleotides or from about 25 to about 200 nucleotides or from about 30 to about 150 nucleotides.

In general, the ASO may comprise a “mismatch motif” or “mismatch region”, which refers to a portion of the ASO sequence that does not have 100% complementarity to its target sequence. An ASO may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The nucleotides of the ASO may or may not be chemically modified. The modification may improve stability of the ASO, such as increase nuclease resistance in addition to protection provided by the LNP. Moreover, in some examples of the disclosure, the chemical modification improves potency and/or selectivity by increasing binding affinity of the ASO with its complementary sequences. Examples of optional modifications include, but are not limited to, 2′-O-alkyl modifications such as 2′-O-Me or 2′-O-methoxyethyl modifications and 2′-halogen modifications such as 2′-fluoro modifications. In yet further embodiments, the ASO comprises one, two, three, four, or more 2′-deoxy nucleotides, e.g., in the sense and/or antisense strand of the double-stranded region. Alternately or additionally, in some embodiments, the ASO comprises phosphate backbone modifications, such as a phosphorothioate backbone modification. Additional backbone modifications include backbone analogues such as locked nucleic acid (LNA). A non-limiting example is a structure that contains a methylene bridge between the 2′ and 4′ positions of the ribose, which “locks” the ribose ring in a conformation that facilitates binding to a complementary nucleic acid sequence. A related bridge modification is a bridged nucleic acid (BNA). Further examples include ASOs with a peptide backbone (PNA), CpG oligomers, among others known to those of skill the art.

The ASO encapsulated in the LNP is generally single-stranded. However, in some examples of the disclosure, the ASO has self-complementary sequences. In such embodiments, the ASO may form one or more stem loop or hairpin structures within the strand.

In some embodiments, the ASO is covalently bound to one or more other moieties to form a conjugate. In some embodiments, the conjugates are selected based on their ability to facilitate delivery of the ASO to an organism or into cells. An ASO may be bound to a moiety at, for example, the 5′ end of the antisense strand, the 3′ end of the antisense strand, the 5′ end of the sense strand, the 3′ end of the sense strand, or to a nucleotide at a position that is not at the 3′ end or 5′ end of either strand.

Examples of conjugates include but are not limited to one or more of an antibody or fragments thereof, a peptide, an amino acid, an aptamer, a phosphate group, a cholesterol moiety, a lipid, a cell-penetrating peptide, a polymer, such as a hydrophilic polymer, such as polyethylene glycol, and a sugar group, which includes a sugar monomer, an oligosaccharide and modifications thereof. In one non-limiting example, the conjugate is N-Acetylgalactosamine (GalNAc).

Methods of designing antisense oligonucleotides are known in the art and can be readily adapted to produce an antisense oligonucleotide that targets any polynucleotide sequence. Selection of antisense oligonucleotide sequences specific for a given target sequence may be based upon analysis of the chosen target sequence and determination of secondary structure, Tₘ, binding energy, and relative stability. The ASO sequence can be arrived at by computational design or by experimentation.

### Vector DNA

The lipid nanoparticle described herein may comprise encapsulated DNA vector. As used herein, the term “DNA vector” refers to a polynucleotide that encodes at least one peptide, polypeptide or protein and that is either circular or has been linearized.

The DNA vector may replicate autonomously, or it may replicate by being inserted into the genome of the host cell, by methods well known in the art. Vectors that replicate autonomously will have an origin of replication or autonomous replicating sequence (ARS) that is functional in in a host cell. The DNA vector is usable in more than one host cell, e.g., in E. coli for cloning and construction, and in a mammalian cell for expression.

The DNA vectors may be administered to a subject for the purpose of repairing, enhancing or blocking or reducing the expression of a cellular protein or peptide. Accordingly, the nucleotide polymers can be nucleotide sequences including genomic DNA, cDNA, or RNA.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will preferably have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acid in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16 (11): 1426-8, which is incorporated herein by reference.

The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present method can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Detailed descriptions of the procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

In another embodiment, the DNA vector is a nanoplasmid or a minicircle.

The DNA vector may be part of a CRISPR/Cas9 or zinc finger nuclease gene editing system. In another embodiment, the DNA vector is used in a diagnostic application.

mRNA

The lipid nanoparticle described herein may comprise a cargo that is messenger RNA. As used herein, the term “messenger RNA” or “mRNA”, refers to a polynucleotide that encodes and expresses at least one peptide, polypeptide or protein. The term is meant to include, but is not limited to, mRNA that is circular or linear as well as small activating RNA (saRNA) and trans-amplifying RNA (taRNA).

The concentration of mRNA in the LNP may be between 0.01 and 20 mg/mL or between 0.01 and 10 mg/mL or between 0.05 and 5 mg/mL or between 0.075 and 4 mg/mL.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is chemically synthesized, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to formulate and encapsulate mRNAs of a variety of lengths. In some embodiments, the present disclosure may be used to formulate and encapsulate in vitro synthesized mRNA ranging from about 1-20 kb, about 1-20 kb, about 1-15 kb, about 1-10 kb, about 2-20 kb, about 2-15 kb, about 2-10 kb, about 5-20 kb, about 5-15 kb, about 5-12 kb, about 5-10 kb, about 8-20 kb, or about 8-15 kb in length.

In those embodiments in which the mRNA is linear, the synthesis includes the addition of a “cap” on the 5′ end, and a “tail” on the 3′ end. The presence of the cap provides resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In a further embodiment, the mRNA is circular. Advantageously, such mRNA lacks 5′ and 3′ ends and thus may be more stable in vivo due to its resistance to degradation by exonucleases. The circular mRNA may be prepared by any known method, including any one of the methods described in Deviatkin et al., 2023, “Cap-Independent Circular mRNA Translation Efficiency”, Vaccines, 11 (2), 238, which is incorporated herein by reference. Translation of the circular mRNA is carried out by a cap-independent initiation mechanism.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional and typically omitted. Such sequences are incorporated into mRNA for in vivo studies in animal models to assess biodistribution.

### Editing Cargo

In one embodiment, the LNP-encapsulated cargo edits a cell to produce a desired modification to treat, prevent or ameliorate a disease or condition.

As used herein, the term “editing cargo” includes a protein and/or nucleic acid-based cargo that causes modification of a cell at a specific locus or loci to produce a desired modification to treat, prevent or ameliorate a disease or condition.

As used herein, the term “nucleic acid editor” includes a protein and/or nucleic acid-based system that causes modification of any nucleic acid of a cell at a specific locus or loci to produce a desired modification to treat, prevent or ameliorate a disease or condition.

The cargo may comprise a nucleic acid that encodes for a protein or peptide that forms part of a nucleic acid editing complex. A “nucleic acid editing complex” includes without limitation protein and/or nucleic acid-based systems in which nucleic acid is inserted, deleted, modified (e.g., epigenetic editing) or replaced in the genetic material of an organism at a site-specific location.

The nucleic acid editing complex may be used for genetic modification of a cell and includes post-translational modifications.

Alternatively or additionally, the cargo comprises a peptide or protein that is part of an editor or forms an editing complex.

The nucleic acid editing complex includes, without limitation, Cas-based (e.g., CRISPR or non-CRISPR), transcription activator-like effector nuclease (TALEN), megaTALs, zinc finger nuclease (ZFN), Adenosine Deaminase Acting on RNA (ADAR), prime editors, base editors, epigenetic, transposase, meganuclease, ARCUS gene editing cargo or any variant or combination thereof. These nucleic acid editing cargo are exemplary and include any cargo that can modify genetic material (including RNA transcripts and non-coding regions) of a cell to treat, prevent or ameliorate a disorder or disease. Without limitation, the gene editing cargo may include those that are designed by a process referred to as Directed Nuclease Editor (DNE), which is known to those of skill in the art.

Cas-based editing cargo comprise CRISPR and non-CRISPR gene editing cargo. In addition, the editing cargo include those that cut DNA as well as epigenetic editing cargo that modify nucleic acid markers, as discussed below.

The CRISPR gene editing cargo most advantageously comprises nucleic acid (e.g., mRNA) encoding for one or more of a Class II Cas nuclease family of proteins and a guide RNA. The nucleases encoded by the nucleic acid are enzymes with DNA endonuclease activity and can be directed to cleave a desired nucleic acid target by an appropriate guide RNA. The nuclease and guide RNA form a complex referred to as a ribonucleoprotein (RNP). In some embodiments, the nuclease is a Class II CRISPR enzyme, which is further subdivided into Types II, V and VI. According to one embodiment, the mRNA encodes for a Cas protein that is part of a Type II CRISPR/Cas system, such as a Cas9 protein or a Cpf1 protein.

In another embodiment, the mRNA encodes for a Cas protein that is part of a Type V CRISPR/Cas system, such as Cas12a. In another embodiment, the mRNA encodes for a Cas protein that is a Cas 13a, which is an RNA endonuclease and cleaves single-stranded RNA.

The guide RNA can direct the Cas nuclease to the target sequence on a target nucleic acid molecule, where the guide RNA hybridizes to the target sequence and the Cas nuclease cleaves or modulates the sequence. In some embodiments, the guide RNA binds to a class 2 nuclease, thereby providing specificity of cleavage.

Guide RNAs for the CRISPR/Cas9 nuclease system include CRISPR RNA (crRNA) or tracr RNA (tracr). In some embodiments, the crRNA can include a targeting sequence that is complementary to and hybridizes to a target sequence on a target nucleic acid molecule. The crRNA can also include a flagpole that is complementary to, and hybridize to, a portion of tracrRNA. In some embodiments, the crRNA can correspond to the structure of a naturally-occurring crRNA transcribed from a bacterial CRISPR locus, wherein the targeting sequence acts as a spacer for the CRISPR/Cas9 system. The flagpole corresponds to the part of the repetitive sequence adjacent to the spacer above the CRISPR locus.

The guide RNA of the RNP can target any sequence of interest through the targeting sequence of crRNA. In some embodiments, the targeting sequence of the guide RNA and the target sequence on the target nucleic acid molecule may be 100% complementary. In other embodiments, the targeting sequence of the guide RNA and the target sequence on the target nucleic acid molecule can comprise at least one mismatch.

The length of the targeting sequence may depend on the RNP system and components used. For example, different Cas proteins from different bacterial species have various optimal targeting sequence lengths. Thus, targeting sequences of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length can be included. In some embodiments, the targeting sequence can comprise a length of 18 to 24 nucleotides. In some embodiments, the targeting sequence can comprise 19-21 nucleotides in length. In some embodiments, the targeting sequence can comprise a length of 20 nucleotides.

In some embodiments, the editing system includes Cas1, CaslB, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csbl11, Csb2, Csb3, Csx17, Csx14, Csx10, Csx16, CsaX, Csx3, Csx1, Csx15, Csf1, Csf2, Csf3, Csf4, homologs thereof, or modified versions thereof.

As noted, non-CRISPR, Cas-based gene editing cargo are encompassed by embodiments of the disclosure as well. A Cas-based editing system may include a Cas enzyme fused to deaminase (Luo et al., 2020, Microbial Cell Factories, 19 (93), incorporated herein by reference). An example is a cytosine base editor or an adenine base editor produced by fusing endonuclease Cas to cytosine deaminase pmCDA1 or heterodimer adenine deaminase TadA-TadA. A further non-limiting example is Cas fused to reverse transcriptase (Mohr et al., 2018, Mol Cell., 72 (4): 700-714, incorporated herein by reference).

Fanzor is a eukaryotic RNA-guided endonuclease that could function as a gene editor in certain embodiments herein. (See Saito et al., 2023, Nature 620:660-668, which is incorporated herein by reference). In some embodiments, Fanzor proteins use RNA as a guide to target DNA precisely and can be modified to edit a cell using LNPs described herein. In some examples, the compact Fanzor cargo may have the ability to facilitate more improved delivery than CRISPR-Cas cargo.

In those embodiments in which the cargo is a TALEN, the cargo comprises a nucleic acid encoding a peptide having a Transcription activator-like (TAL) effector DNA binding domain, a fragment or a variant thereof. In an embodiment, the system comprises a nucleic acid encoding a peptide having nuclease activity, e.g., endonuclease activity. In an embodiment, the peptide having nuclease activity is a type-II restriction 1-like endonuclease, e.g., a Fokl endonuclease.

In those embodiments in which the cargo is a ZFN, the nucleic acid may encode a peptide having: a Zinc finger DNA binding domain, a fragment or a variant thereof; and/or nuclease activity, e.g., endonuclease activity. In an embodiment, the Zn finger binding domain comprises 1, 2, 3, 4, 5, 6, 7, 8 or more Zinc fingers. In an embodiment, the peptide having nuclease activity is a type-II restriction 1-like endonuclease, e.g., a Fokl endonuclease.

Adenosine Deaminase Acting on RNA (ADAR) is another editing cargo encompassed by embodiments of the disclosure that may be used for post-transcriptional modification of RNA. Examples include ADAR1 and ADAR2. ADAR1 may catalyze posttranscriptional deamination of C6 of adenosines in dsRNA, converting them to inosines (see Song et al., 2022, PMC, 13 (1): e1665, incorporated herein by reference).

Meganucleases are enzymes in the endonuclease family that may induce homologous recombination, generate mutations and alter reading frames. The meganuclease includes homing endonucleases that are intron or intein endonucleases. In one embodiment, the meganuclease is from the LAGLIDADG family, a GIY-YIG endonuclease, an HNH endonuclease, a His-Cys box endonuclease or a PD-(D/E) XK endonuclease. Meganucleases may be combined with components of other gene editing system. In one embodiment, a DNA binding domain from a transcription activator-like (TAL) effector is combined with a meganuclease to produce a “megaTAL”. In another embodiment, a meganuclease may be fused to a DNA end-processing enzyme to promote an error-prone non-homologous end joining.

ARCUS nuclease is a gene editing system based on I-Crel, which is a kind of homing endonuclease that evolved in the algae Chlamydomonas reinhardtii. In some embodiments, the nuclease can deactivate itself after gene editing, thereby reducing off-targeting. ARCUS nucleases in some embodiments can generate a unique cleavage site that is a four-base-pair, 3′ overhang and may be able to carry out gene insertion, gene excision, gene repair or a combination thereof.

Epigenetic editing is also encompassed by examples of the disclosure. Such editing of genetic material does not cut nucleic acid but rather alters epigenomic marks “adorning” DNA. Changing the epigenic signature of a cell can serve to modify an epigenetic signature of the cell and change its transcriptional profile. In some embodiments, the epigenetic editing system may target and edit one or more methylation sites of a nucleic acid sequence. In some embodiments, genome homing proteins with engineered or naturally occurring nuclease functions for gene editing, can be mutated and adapted to function as only delivery systems. In one embodiment, an epigenetic modifying enzyme or domain can be fused to the homing protein and local epigenetic modifications can be altered upon protein recruitment. A targeting protein that recognizes DNA sequences may be linked to an effector protein that alters epigenomic marks, such as methylation. Examples of targeting proteins include Transcription Activator-Like Effector (TALE), zinc finger proteins, and Cas systems, including but not limited to CRISPR-Cas. Non-limiting examples of effector proteins include TET1, which induces demethylation of cytosine at CpG sites; LSD1, which induces demethylation of H3K4me1/2, which also causes an indirect effect of deacetylation on H3K27; and CIB1/CRY2, which is a cryptochrome/blue light activated complex allowing chromatin to be modified upon illumination.

Further examples of effector proteins include DNA methyltransferase, a fragment (e.g., a biologically active fragment) or variant thereof (e.g, DNMT1, DNMT2 DNMT3A, DNMT3B, DNMT3L, or CpG methyltransferase (M. Sssl)); or a poly comb repressive complex or a component thereof, e.g, PRC1 or PRC2, or PR-DUB, or a fragment (e.g, biologically active fragment) or a variant thereof.

In an embodiment, the epigenetic editor comprises a molecule that modifies chromatin architecture and/or modifies a histone. In an embodiment, the epigenetic modulator is a molecule that modifies chromatin architecture, e.g, a SWI/SNF remodeling complex or a component thereof. In an embodiment, the epigenetic modulator is a molecule that modifies a histone, e.g, methylates and/or acetylates a histone, e.g, a histone modifying enzyme or a fragment (e.g, biologically active fragment) or a variant thereof, e.g, HMT, HDM, HAT, or HD AC.

Improved Targeted Expression and Biodistribution of Ligand-LNPs with Increasing Neutral Lipid Content

As described in the Example section, the targeting moiety-LNPs of the disclosure having elevated neutral lipid content may provide improved biodistribution to a wider range of tissues and/or organs than an Onpattro™-type formulation or a baseline formulation described herein. In a further embodiment, the baseline formulation may be (a) an otherwise identical LNP having 10 mol % lower levels of the same neutral lipid; (b) when the N/P of the LNP is 4 or greater, an otherwise identical LNP having an N/P that is 1 or 3; and/or (c) when the lipid nanoparticle has a weight nucleic acid/micromole of total lipid that is 0.05:1 to 1:1, an otherwise identical LNP having a weight nucleic acid/micromole of total lipid that is 0.20:1 less than that of the lipid nanoparticle of the disclosure.

The LNP of the disclosure in one embodiment exhibits increased biodistribution in the liver, spleen, bone marrow, heart, lung, kidney, abdominal skin, back skin and/or ear in a specified mouse model than the relevant baseline. In another embodiment, this includes increased biodistribution to extrahepatic tissues selected from the spleen, bone marrow, heart, lung, kidney, abdominal skin, back skin and/or ear relative to the relevant baseline. In another embodiment, this includes increased biodistribution to extrahepatic tissues selected from the spleen or bone marrow relative to the relevant baseline. Whether or not an LNP encapsulating oligonucleotide exhibits such enhanced biodistribution to one or more of a given tissue or organ relative to a baseline oligo-LNP formulation is determined by biodistribution studies in an in vivo mouse model as detailed in the Example section. A fluorescent lipid marker (DiD as described in the Materials and Methods) is used to assess biodistribution of the oligo-LNP in a given tissue or organ relative to the baseline.

In one embodiment, the lipid nanoparticle exhibits at least a 5%, 10%, 20%, 30%, 40%, 50%, 60%, 70%, 80%, 90%, 100%, 110%, 120%, 130%, 140%, 150%, 160%, 170%, 180%, 190% or 200% increase in biodistribution as measured in vivo in the liver, spleen, bone marrow, heart, lung, kidney, abdominal skin, back skin and/or ear of a mouse relative to any one of the above-described relevant baselines, wherein the biodistribution is measured in a mouse model by detection of a lipid marker at 1, 4, 10 and/or 24 hours post-administration. The measurement is carried out on tissue homogenates of one or more of the foregoing tissues or organs as set forth in the Example section.

The percentage increase in fluorescence relative to the relevant baseline is determined by comparing the fluorescence signal of an LNP being assessed in a relevant tissue and/or organ per mg of tissue homogenate to a tissue homogenate fluorescent signal resulting from administration of a baseline LNP.

The oligo-LNPs being compared are prepared using identical materials and methods. In other words, the two formulations compared have the same ionizable lipid, PEG-lipid (if included) and sterol and are prepared using rapid ethanol injection as set out in the Materials and Methods.

The biodistribution is assessed at the same time point post-administration (1, 4, 10 and/or 24 hours) to the same mice and using the same analytical technique to measure the marker lipid (see Materials and Methods).

In those embodiments in which the baseline formulation has 10 mol % less neutral lipid (e.g., DSPC or sphingomyelin) than the LNP of the disclosure, the neutral lipid may be decreased in the baseline at the expense of both cholesterol and ionizable lipid in equal proportions but keeping the ionizable lipid:cholesterol (mol:mol) constant between baseline and LNP of the disclosure.

### Clinical and Non-Clinical Uses of the LNP Herein

In some embodiments, the LNP encapsulating nucleic acid is part of a pharmaceutical composition and is administered to treat and/or prevent a disease condition. The treatment may provide a prophylactic (preventative), ameliorative or a therapeutic benefit. The pharmaceutical composition will be administered at any suitable dosage.

The nucleic acid-LNPs described herein may be used to treat and/or prevent any disease, disorder or condition in a mammalian subject. This includes a disease, disorder or condition, such as cancer, infectious diseases such as bacterial, viral, fungal or parasitic infections, inflammatory and/or autoimmune disorders, including treatments that induce immune tolerance and cardiovascular diseases such as hypertension, cardiac arrhythmia and restenosis.

Examples of cancers include lung cancer, colon cancer, rectal cancer, anal cancer, bile duct cancer, small intestine cancer, stomach (gastric) cancer, esophageal cancer; gallbladder cancer, liver cancer, pancreatic cancer, appendix cancer, breast cancer, ovarian cancer; cervical cancer, prostate cancer, renal cancer (e.g., renal cell carcinoma), cancer of the central nervous system, glioblastoma, skin cancer, lymphomas, choriocarcinomas, head and neck cancers, osteogenic sarcomas, and blood cancers. Non-limiting examples of specific types of liver cancer include hepatocellular carcinoma (HCC), secondary liver cancer (e.g., caused by metastasis of some other non-liver cancer cell type), and hepatoblastoma.

Non-limiting examples of other diseases, disorders or conditions that may be treated by the oligo-LNPs herein and that may be attributed at least in part to an immunological disorder include colitis, Crohn's disease, allergic encephalitis, allograft transplant/graft vs. host disease (GVHD), diabetes and multiple sclerosis.

The LNPs herein may also be used in other applications besides the treatment and/or prevention of a disease or disorder. The LNPs may be used to treat conditions such as aging, preventative medicine and/or as part of a personalized medicine regime. In further embodiments, the LNP is used in a diagnostic application.

In one embodiment, the LNP is part of a pharmaceutical composition administered parenterally, i.e., intra-arterially, intravenously, subcutaneously or intramuscularly. In yet a further embodiment, the pharmaceutical compositions are for intra-tumoral administration. In another embodiment, the pharmaceutical compositions are administered intranasally, intravitreally, subretinally, intrathecally or via other local routes. In some embodiments, the oligo-LNP is applied or administered to the skin.

The pharmaceutical composition comprises pharmaceutically acceptable salts and/or excipients. As used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

As used herein, the term “excipient” means the substances used to formulate active pharmaceutical ingredients (API) into pharmaceutical formulations. Non-limiting examples include mannitol, Captisol®, lactose, starch, magnesium stearate, sodium saccharine, talcum, cellulose, sodium crosscarmellose, glucose, gelatin, sucrose, magnesium carbonate, and the like. Acceptable excipients are non-toxic and may be any solid, liquid, semi-solid excipient that is generally available to those of skill in the art.

The compositions described herein may be administered to a subject. The term subject as used herein includes a human or a non-human subject, including a mammal. The oligo-LNP may be administered as part of a preventative treatment and so the subject is not limited to a patient.

The examples are intended to illustrate the preparation of specific lipid nanoparticle oligo preparations and properties thereof but are in no way intended to limit the scope of the invention.

The article “a” or “an” as used herein is meant to include both singular and plural forms of the term or phrase being referred to herein, unless otherwise indicated.

### EXAMPLES

### Methods and Materials

### Preparation of Surface Modified Lipid Nanoparticles Containing Nucleic Acid

Unless otherwise specified, the LNPs were prepared by dissolving mRNA or plasmid DNA (pDNA) in 25 mM sodium acetate, pH 4.0, while the lipid components at the mole % specified were dissolved in absolute ethanol. The lipids in ethanol and the nucleic acid cargo in buffer were combined in a 1:3 volume by volume ratio using a t-junction with dual-syringe. The solutions were pushed through the t-junction at a combined flow rate of 20 mL/min (5 mL/minute for the lipid-containing syringe, 15 mL/minute for the mRNA-containing syringe). The mixture was subsequently dialyzed overnight against at least ~100 volumes of 1× phosphate buffered saline (PBS), pH 7.4 using Spectro/Por™ dialysis membranes (molecular weight cut-off 12 000-14 000 Da). The LNPs were concentrated as required with an Amicon Ultra™ 10 000 MWCO (molecular weight cut-off), regenerated cellulose concentrator. The targeting moiety-lipophilic moiety was an arginine-glycine-aspartate (RGD) peptide conjugated DSPE-PEG lipid. The ionizable lipid was nor-MC3 (described in WO 2022/246571, incorporated herein by reference). The remaining lipids include 1,2-dimyristoyl-rac-glycero-3-methoxypolyethylene glycol-2000 (DMG-PEG2k), distearoylphosphatidylcholine (DSPC) and cholesterol (Chol).

Encapsulation efficiency was calculated by determining unencapsulated mRNA content by measuring the fluorescence upon the addition of RiboGreen™ to the mRNA-LNP (Fᵢ) and comparing this value to the total mRNA content that is obtained upon lysis of the LNP by 2% Triton X-100 (Fₜ): % encapsulation=(Fₜ−Fᵢ)/Fₜ×100.

The particle size and polydispersity index (PDI) were characterized using a Zetasizer Nano ZS™.

### In Vitro Analysis in A7 Astrocyte Cells

A7 Astrocyte cells were cultured in Dulbecco's Modified Eagle Medium (DMEM) supplemented with 10% Fetal Bovine Serum (FBS). For cell treatments, 10,000 cells were added to each well in a 96-well plate. 24 hours later, the medium was aspirated and replaced with medium containing diluted LNP at the relevant concentration over a range of 0.03-10 μg/mL mRNA. Expression analysis was performed 24 hours later, and luciferase levels measured using the Steady-Glo Luciferase kit (Promega). Cells were lysed using the Glo Lysis buffer (Promega).

### Tissue Homogenate Assay

Tissues were removed from the mice and placed in 2 mL tubes and snap frozen in liquid nitrogen. The tissues were subsequently frozen at −80° C. An appropriate volume of GLO™ lysis buffer from Promega™ was added to each of the tubes, ensuring that the samples remained frozen before addition of the lysis buffer. Samples were placed in a FastPrep™ homogenizer and the homogenizer was operated and repeated 2 times for a total of three rounds. The homogenized samples were centrifuged at room temperature and subsequently homogenate was added to a black plate. The plate was transferred to a plate reader and the fluorescence was read at 640 nm excitation/720 nm emission.

### Cryo-TEM

The following describes a method to assess the morphology of LNPs. LNPs are concentrated to between 15-25 mg/mL estimated total lipid prior to cryo-TEM imaging. A defined volume, for example 2-4 μL, of the resulting LNP solution is applied to a glow-discharged copper grid, and plunge-frozen using an FEI Mark IV Vitrobot to generate vitreous ice. These grids are stored in liquid nitrogen until imaged by an FEI Titan Krios or an FEI Glacios TEM. The instrument is operated at 200 kV in low-dose conditions and the resulting images are obtained using a bottom-mount FEI Falcon direct electron detector camera at 47-88,000× magnification with an under-focus of 0.5-2 μm in order to enhance contrast.

### Example 1: Particle Characteristics of LNPs Having Elevated Neutral Lipid and Modified with a Targeting Moiety

The encapsulation efficiency, PDI and particle size of the following lipid nanoparticles A-E with varying amounts of RGD-PEG-lipid and different types of sterol (cholesterol and beta-sitosterol) were measured (see Materials and Methods). All formulations tested contained elevated levels of neutral lipid (40 mol % DSPC). Formulations A-D contained cholesterol as the sterol and included varying mol % of the targeting moiety lipid conjugate (RGD modified). Formulation E contained beta-sitosterol as the sterol and 1.5 mol % of the targeting moiety lipid conjugate (RGD modified). The ionizable lipid was norMC3 (described in WO 2022/246571).

TABLE 1
Targeting moiety modified LNPs with elevated neutral lipid
examined for physiochemical particle characteristics

| | Targeting | |
|---|---|---|
| LNP | moiety-lipid | Compositions in mol % of ionizable lipid:neutral |
| identi- | (RGD-PEG- | lipid:sterol:PEG-lipid or PEG-lipid with RGD |
| fier | lipid) mol % | targeting moiety |
| A | 0 | nMC3-37.4%:40% DSPC:Chol |
| | | 21.1%:1.5% DMG-PEG2k |
| B | 1 | nMC3 37.9%:40% DSPC:Chol |
| | | 21.1%:DSPE-PEG2k-RGD 1% |
| C | 2 | nMC3 36.9%:40% DSPC:Chol |
| | | 21.1%:DSPE-PEG2k-RGD 2% |

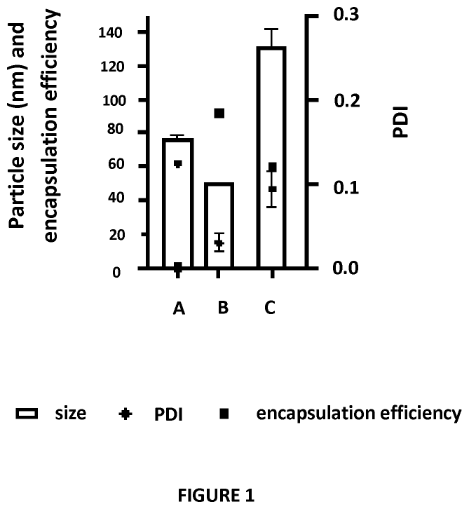

The results are shown in FIG. 1 and suggest that, in non-limiting embodiments of the disclosure, the targeting moiety-lipophilic moiety included in the particles at less than 2 mol % achieves suitable PDI, particle size and encapsulation efficiency.

### Example 2: Dose Dependent In Vitro Activity of LNPs Having Elevated Neutral Lipid and Modified with a Targeting Moiety

The following lipid nanoparticles G, H and I in Table 2 below were analyzed for dose dependent activity in A7 Astrocyte cells as per the Materials and Methods.

TABLE 2
Targeting moiety modified LNPs with elevated
neutral lipid examined for in vitro activity

| | Targeting | |
|---|---|---|
| | moiety-lipid | Compositions in mol % (ionizable lipid:neutral |
| | (RGD-PEG- | lipid:Chol:PEG-lipid or PEG-lipid with RGD |
| LNP | lipid) mol % | targeting moiety) |
| D | 0 | nMC3 37.4%:40% DSPC:Chol |
| | | 21.1%:1.5% DMG-PEG2k |
| E | 0.5 | nMC3 38.4%:40% DSPC:Chol |
| | | 21.1%:DSPE-PEG-RGD 0.5% |

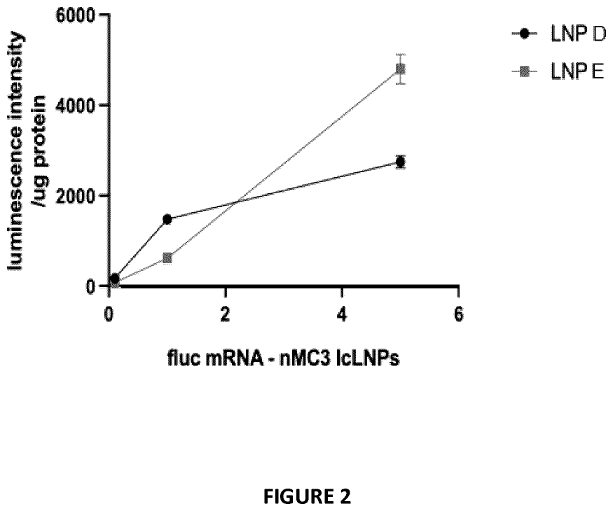

The results are shown in FIG. 2 and suggest that, in non-limiting embodiments of the disclosure, the targeting moiety modified LNPs exhibited enhanced transfection as measured in the Astrocyte cells.

### Example 3: Tissue Expression of mRNA-LNPs Having Targeting Moiety and Elevated Neutral Lipid Content

The in vivo expression of the lipid nanoparticles B and C of Table 1 (see Example 1) with varying amounts of RGD-PEG-lipid was determined (see Materials and Methods) in mice.

All formulations tested contained elevated levels of neutral lipid (40 mol % DSPC) and 1 mol % and 2 mol % of RGD-PEG-lipid. The formulations contain nMC3 37.9%: 40% DSPC: Chol 21.1%:DSPE-PEG2k-RGD 1% (LNP B) and nMC3 36.9%: 40% DSPC: Chol 21.1%:DSPE-PEG2k-RGD 2%.

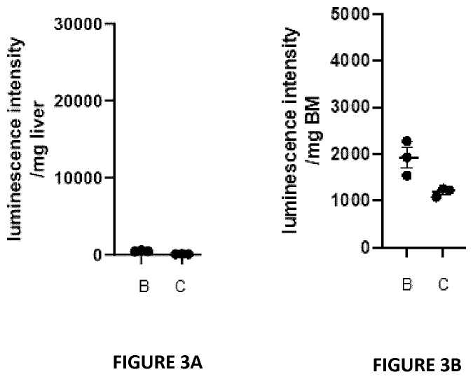

Tissue homogenates of liver and bone marrow were analyzed and the results are shown in FIGS. 3A and 3B, respectively.

Notably, both formulations B and C exhibited increased expression of mRNA in the bone marrow versus the liver. These results show that LNPs having elevated neutral lipid and targeting moiety at both 1 mol % and 2 mol % exhibit enhanced extrahepatic expression as measured in vivo.

### Example 4: Expression of mRNA-LNPs Having Elevated Neutral Lipid Content and Targeting Moiety Against Cell Surface Markers of Haematopoietic Stem and Progenitor Bone Marrow Cells

The effect of modifying an LNP having 50 mol % DSPC with an antibody against CD117 was evaluated for expression of cargo in bone marrow haematopoietic stem and/or progenitor cells (HSPCs) in vivo. As a negative control, the same LNPs were prepared with an antibody against a cell surface marker for T-cells (anti-CD5). The model used was of C57Bl/6 mice.

In particular, the following eGFP mRNA formulations (reported in mol %) comprising nMC3 ionizable lipid (WO 2022/246571), DSPC, cholesterol, PEG-lipid and antibody-conjugated lipid were compared in this example.

TABLE 3
Formulations examined in vivo containing eGFP mRNA

| | | Percent | |
|---|---|---|---|
| Group | Sample | DSPC | Lipid composition/mol % |
| A | PBS | NA | NA |
| B | Onpattro ™ | 10 mol % | nMC3:DSPC:Chol:PEG₂₀₀₀-DMG |
| | | | (50:10:38.5:1.5) |
| C | IcLNP ™ | 50 mol % | nMC3:DSPC:Chol:PEG₂₀₀₀-DMG |
| | | | (27.4:50:21.1:1.5) |
| D | aCD117- | 50 mol % | nMC3:DSPC:Chol:-aCD117- |
| | IcLNP ™ | | PEG₂₀₀₀-DMG (27.4:50:21.1:1.5) |
| E | aCD5 | 50 mol % | nMC3:DSPC:Chol:aCD5- |
| | IcLNP ™ | | PEG₂₀₀₀-DMG (27.4:50:21.1:1.5) |

The gating schemes were as follows:

TABLE 4
Gating scheme on live haematopoietic
stem and progenitor (HSPC) cells

| HSPC Population | | |
|---|---|---|
| in Bone Marrow | Gating Scheme | Figure |
| LK | Lineage⁻c-Kit⁺ | 4A |
| LSK | Lineage⁻ c-Kit⁺ Sca1⁺ | 4A |
| MPPs | Lineage⁻ ckit⁺ Sca1⁺ CD34⁺ | 4B |
| ST-HSCs | Lineage⁻ ckit⁺ Sca1⁺ CD34⁻ | 4B |
| | CD135⁺ | |
| Long-term HSC (LT-HSC) | Lineage⁻ ckit⁺ Sca1⁺ CD34⁻ | 4B |
| | CD135⁻ | |
| Long-term HSC (LT-HSC) | Lineage ckit⁺ Sca1⁺ CD34⁻ | 4C |
| | CD135⁻ CD48⁻ CD150⁺ | |

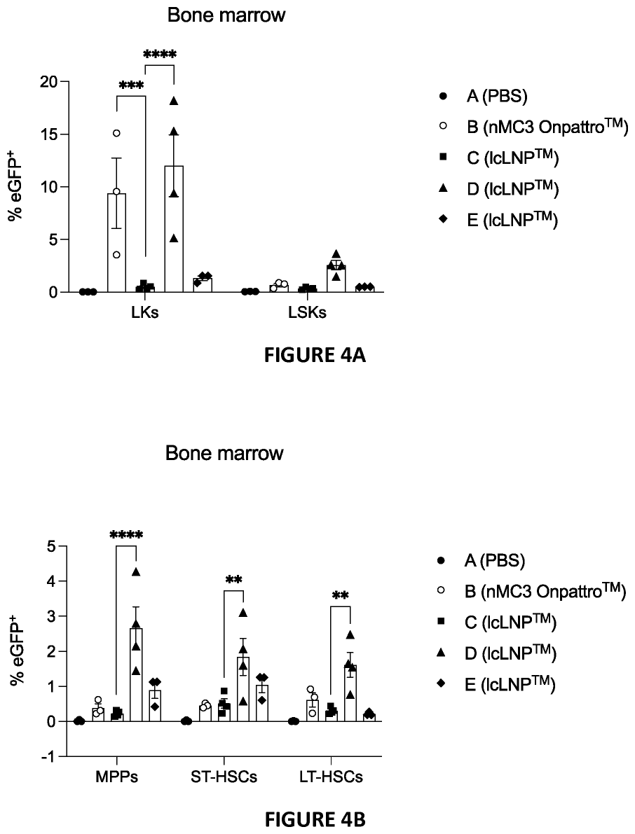

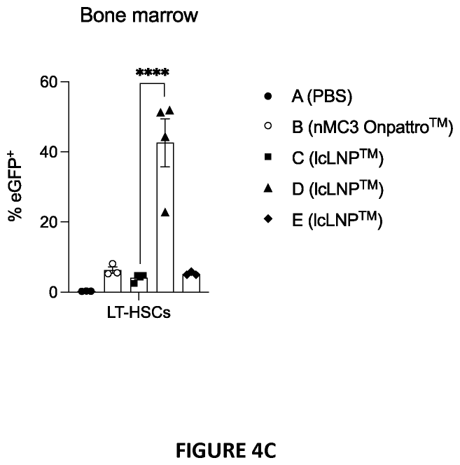

For FIGS. 4A and 4B, statistics were determined with 2-way anova, compared to a control group of LNP C (nMC3 lcLNP™). For FIG. 4C, statistics were determined with 1-way anova, compared to a control group of LNP C (nMC3 lcLNP™).

The results show that Formulation D (aCD117-lcLNP™) with antibody against the CD117 cell surface marker in haematopoietic cells exhibited a higher eGFP expression relative to both Onpattro, lcLNP without the antibody and the aCD5 against T-cell markers (FIG. 4A-C).

The specification is intended to illustrate embodiments and examples of the invention but is in no way intended to limit the scope of the invention as defined by the appended claims.

The article “a” or “an” as used herein is meant to include both singular and plural, unless otherwise indicated.

1. A lipid nanoparticle comprising:
(i) a nucleic acid;
(ii) a neutral lipid content of greater than 35 mol %;
(iii) an ionizable, cationic lipid content of from 5 mol % to 50 mol %;
(iv) a sterol or a derivative thereof; and
(v) a targeting moiety linked to a lipophilic moiety that is present in a lipid layer of the nanoparticle, the targeting moiety optionally linked to the lipophilic moiety via a linker,
wherein each mol % is relative to a total lipid content of the lipid nanoparticle,
and optionally wherein the lipid nanoparticle comprises a core, the core comprising an electron dense region and an aqueous portion, and wherein the core is surrounded at least partially by the lipid layer, as visualized by cryogenic electron microscopy (cryo-EM).

2. The lipid nanoparticle of claim 1, wherein the linker is a hydrophilic polymer that is conjugated at its one end to the lipophilic moiety and at its other end to the targeting moiety.

3. The lipid nanoparticle of claim 1, wherein the lipid nanoparticle is produced by ethanol injection comprising a step of lowering the pH of a solution external to the nanoparticle after its formation, thereby producing the core comprising the electron dense region and the aqueous portion.

4. The lipid nanoparticle of claim 1, wherein the phosphatidylcholine lipid is distearoylphosphatidylcholine (DSPC), dioleoylphosphatidylcholine (DOPC), 1-palmitoyl-2-oleoyl-phosphatidylcholine (POPC), dimyristoyl-phosphatidylcholine (DMPC) or dipalmitoyl-phosphatidylcholine (DPPC).

5. (canceled)

6. (canceled)

7. (canceled)

8. (canceled)

9. (canceled)

10. (canceled)

11. (canceled)

12. The lipid nanoparticle of claim 1, wherein the cationic lipid is an amino lipid.

13. (canceled)

14. The lipid nanoparticle of claim 1, further comprising a hydrophilic polymer-lipid conjugate that is present at a lipid content of 0.5 mol % to 5 mol %.

15. (canceled)

16. (canceled)

17. The lipid nanoparticle of claim 1, wherein the lipid nanoparticle exhibits at least a 10% increase in biodistribution in the liver, spleen, bone marrow, heart, lungs, kidney, abdominal skin, back skin and/or ear as compared to a baseline formulation without the targeting moiety but otherwise identical and/or an Onpattro-type formulation encapsulating the nucleic acid, but otherwise measured under an identical set of conditions, and wherein the biodistribution is quantified in an animal model by detection of a labelled lipid at 24 hours post-administration.

18. The lipid nanoparticle of claim 1, wherein the lipid nanoparticle exhibits at least a 10% increase in mRNA expression in the liver, spleen, bone marrow, heart, lungs, kidney, abdominal skin, back skin and/or ear as compared to a baseline formulation without the targeting moiety but otherwise identical and/or an Onpattro-type formulation encapsulating the nucleic acid and wherein the biodistribution is quantified in an animal model by detection of a labelled lipid at 24 hours post-administration.

19. The lipid nanoparticle of claim 1, wherein the lipid nanoparticle exhibits at least a 10% increase in biodistribution in the spleen, bone marrow, heart, lungs, kidney, abdominal skin, back skin and/or ear as compared to a baseline formulation without the targeting moiety but otherwise identical and/or an Onpattro-type formulation encapsulating the nucleic acid, but otherwise measured under an identical set of conditions, and wherein the biodistribution is quantified in an animal model by detection of a labelled lipid at 24 hours post-administration.

20. The lipid nanoparticle of claim 1, wherein the lipid nanoparticle exhibits at least a 10% increase in mRNA expression in the spleen, bone marrow, heart, lungs, kidney, abdominal skin, back skin and/or ear as compared to a baseline formulation without the targeting moiety but otherwise identical and/or an Onpattro-type formulation encapsulating the nucleic acid and wherein the biodistribution is quantified in an animal model by detection of a labelled lipid at 24 hours post-administration.

21. The lipid nanoparticle of claim 1, wherein the targeting moiety is present at less than 2 mol %, less than 1.8 mol %, less than 1.5 mol % or less than 1.5 mol %.

22. (canceled)

23. (canceled)

24. (canceled)

25. A method for delivering a nucleic acid to a cell to treat a disease, disorder or condition, the method comprising contacting the lipid nanoparticle of claim 1 with the cell in vivo or in vitro.

26. The method of claim 25, wherein the nucleic acid accumulates in the spleen, bone marrow, heart, lungs and/or kidney of the subject at least one day post-administration.

27. The method of claim 25, wherein the disease, disorder or condition is an autoimmune disorder.

28. The method of claim 25, wherein the disease, disorder or condition is an infectious disease.

29. The method of claim 25, wherein the disease, disorder or condition is cancer.

30. The method of claim 25, wherein the cell is a stem cell.

31. The method of claim 30, wherein the stem cell is a hematopoietic stem or progenitor cell.

32. The method of claim 25, wherein the cell is a T-cell.

33. (canceled)

34. (canceled)

35. A lipid nanoparticle encapsulating nucleic acid and having at least 38 mol % neutral lipid, a sterol or derivative thereof, and a targeting moiety anchored to the lipid nanoparticle via a lipophilic moiety, wherein a linker is optionally present between the lipophilic moiety and the targeting moiety, wherein the lipid nanoparticle comprises a core, the core comprising an electron dense region and an aqueous portion, and wherein the core is surrounded at least partially by the lipid layer, as visualized by cryogenic electron microscopy (cryo-EM).
